(19) United States
(12) Patent Application Publication
(10) Pub. No.: US 2025/0204915 A1
(43) Pub. Date: Jun. 26, 2025

(54) COMPLIANCE-ENHANCING BANDS

(71) Applicant: EDWARDS LIFESCIENCES CORPORATION, Irvine, CA (US)

(72) Inventors: Sakyasingh Tripathy, San Diego, CA (US); Douglas Thomas Dominick, Irvine, CA (US); Hengchu Cao, Irvine, CA (US); Koray Senol, Laguna Niguel, CA (US); Bingquan Su, Irvine, CA (US); Nima V. Nia, Mission Viejo, CA (US); Yousef Naranjani, Lake Forest, CA (US); Sean Timothy O'keeffe, Tustin, CA (US); Arvin T. Chang, Yorba Linda, CA (US); Ashley Lauren Spink, Newport Beach, CA (US); Emil Karapetian, Huntington Beach, CA (US)

(21) Appl. No.: 19/019,231

(22) Filed: Jan. 13, 2025

Related U.S. Application Data
(63) Continuation of application No. PCT/US2023/071199, filed on Jul. 28, 2023.
(60) Provisional application No. 63/370,423, filed on Aug. 4, 2022.

(51) Int. Cl. A61B 17/12; A61B 17/00

(52) U.S. Cl. CPC A61B 17/12013; A61B 2017/00778; A61B 2017/12018

(57) ABSTRACT

A process for adding compliance to a blood vessel preferably includes forming a first puncture through a first wall segment of the blood vessel, forming a second puncture through a second wall segment of the blood vessel at a diametrically opposed location, deploying a first tissue anchor on an outside of the blood vessel in an area of the first puncture, deploying a second tissue anchor on the outside of the blood vessel in an area of the second puncture, and tensioning an elastic band coupled to the first tissue anchor and the second tissue anchor, thereby causing the elastic band to reshape the target segment of the blood vessel to a non-circular cross-sectional shape. The elastic band elongates and contracts during cardiac cycles for adding compliance to the blood vessel.

### RELATED APPLICATION(S)

This application is a continuation of International Patent Application No. PCT/US2023/071199, filed Jul. 28, 2023, which claims the benefit of U.S. Provisional Patent Application Ser. No. 63/370,423, filed on Aug. 4, 2022, the complete disclosures of which are hereby incorporated by reference in its entireties.

### BACKGROUND

### Field

The present disclosure generally relates to the field of medical implant devices and procedures.

### Description of Related Art

Insufficient or reduced compliance in certain blood vessels, including arteries such as the aorta, can result in reduced perfusion, cardiac output, and other health complications. Increasing compliance and/or otherwise controlling flow in such blood vessels can improve patient outcomes.

### SUMMARY

Described herein are devices, methods, and systems that facilitate the restoration and/or enhancement of compliance characteristics for target blood vessels. Devices associated with the various examples of the present disclosure can include one or more energy-storing bands configured to be connected across at least a portion of an inner diameter of the aorta or other target blood vessel to generate vascular compliance. For example, the band(s) can be configured to expand and contract, thereby reshaping the target blood vessel to provide a change in volume of the target blood vessel over the cardiac cycle to mimic compliance of a healthy blood vessel and/or otherwise promote blood flow during, for example, the diastolic phase of the cardiac cycle.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIGS. 1A, 1B, and 1C illustrate example representations of cardiac and vascular anatomy of a patient.

FIGS. 2A and 2C provide cross-sectional and side views, respectively, of a blood vessel experiencing compliant expansion during the systolic phase of the cardiac cycle.

FIGS. 2B and 2D provide cross-sectional and side views, respectively, of a blood vessel experiencing compliant contraction during the systolic phase of the cardiac cycle.

FIGS. 3A and 3B provide cross-sectional and side views, respectively, a blood vessel with reduced compliance.

FIG. 4 is a graph illustrating blood pressure over time in an example healthy patient.

FIG. 5 is a graph illustrating blood pressure over time in an example patient having reduced aortic compliance.

FIGS. 6A, 6B, and 6C show a blood vessel in circular, oval, and peanut shapes, respectively.

FIGS. 7A and 7B show a compliance-enhancing band device implanted in a blood vessel, in contracted and expanded states, respectively, in accordance with one or more examples.

FIG. 8 shows a compliance-enhancing band implant including hemostasis elements in accordance with one or more examples.

FIG. 9 shows a blood vessel having multiple compliance-enhancing implant devices implanted therein in an axially-offset arrangement in accordance with one or more examples.

FIGS. 10A and 10B show an implant device comprising band segments that are joined by a lock/fixation element in accordance with one or more examples.

FIGS. 11A, 11B, 11C, 11D, and 11E illustrate example configurations of band-type compliance-enhancing implant devices comprising four tissue anchors in accordance with one or more examples.

FIGS. 12A, 12B, 12C, 12D, and 12E illustrate example configurations of band-type compliance-enhancing implant devices comprising three tissue anchors in accordance with one or more examples.

FIGS. 13A, 13B, 13C, and 13D illustrates compliance-enhancing band-type implant devices having wheel-and-spoke tissue anchors in accordance with one or more examples.

FIGS. 14A and 14B illustrate a flow diagram for a process for implanting a compliance-enhancing band implant device using a minimally-invasive access in accordance with one or more examples.

FIGS. 15A and 15B provide images of the compliance-enhancing implant device and certain anatomy corresponding to operations of the process of FIGS. 14A and 14B according to one or more examples.

FIGS. 16A and 16B illustrate a flow diagram for a process for implanting a compliance-enhancing band implant device using a transcatheter access in accordance with one or more examples.

FIGS. 17A and 17B provide images of the compliance-enhancing implant device and certain anatomy corresponding to operations of the process of FIGS. 16A and 16B according to one or more examples.

FIGS. 18A, 18B, 18C, 18D, and 18E show configurations of a compliance-enhancing band implant device comprising a single integrated coil device in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, compliance-enhancement implant devices implanted in the aorta. However, although certain principles disclosed herein may be particularly applicable to the anatomy of the aorta, it should be understood that compliance-enhancement implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

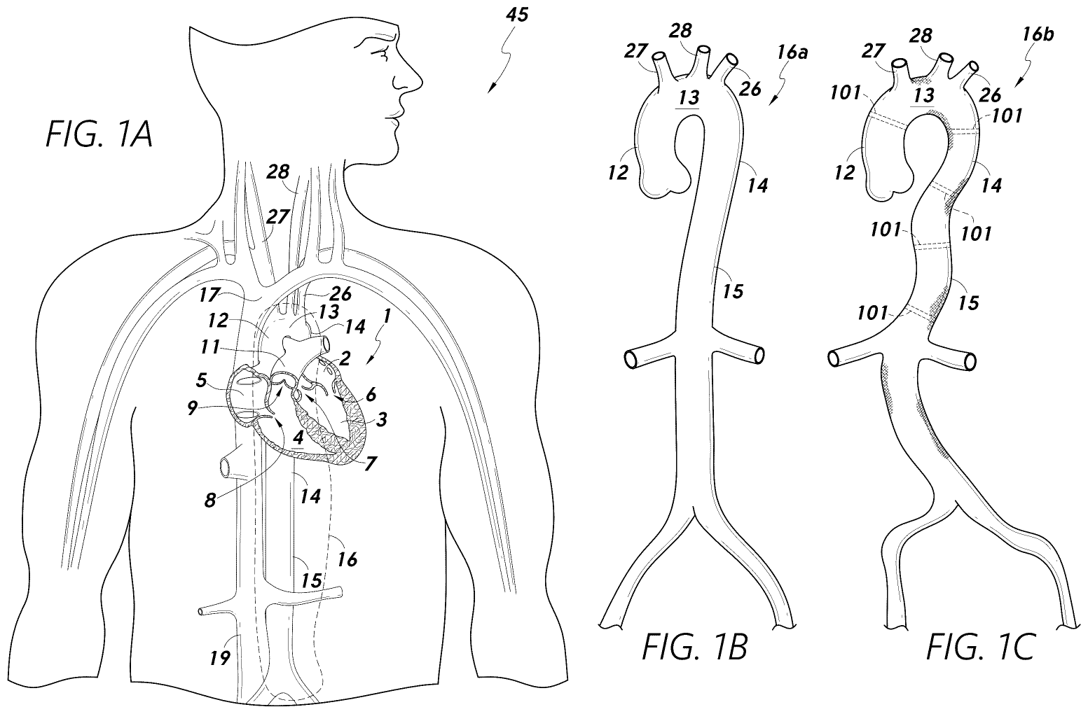

FIG. 1A illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the ascending aortic trunk 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior and superior venae cavae, carry blood back to the heart.

FIGS. 1B and 1C show detailed views of example healthy 16a and aged/stiff 16b aortas, respectively. The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart and is sometimes referred to as the aortic ‘trunk.’ The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively frequently affected by aneurysms and dissections, often requiring open heart surgery to be repaired. The transition from the ascending aorta 12 to the aortic arch 13 is at the pericardial reflection on the aorta 16. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions (e.g., lesser volume) as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

A healthy aorta 16a, as shown in FIG. 1B, runs along a generally straight path, whereas an aged and/or stiffened aorta 16b, as shown in FIG. 1C, can run along a more tortuous, curved path. That is, the aorta tends to change in shape as a function of age, resulting in higher degrees of curvature or tortuosity, as developed gradually over time. Such change in shape of the blood vessel can be associated with the vasculature of the subject becoming less elastic. As such conditions develop, arterial blood pressure (e.g., left-ventricular afterload) can become more pulsatile, which can have deleterious effects, such as the thickening of the left ventricle (LV) muscle, and insufficient perfusion of the heart, Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles, and peripheral edema, for example, which may be caused by structural and/or functional cardiac abnormality. Such conditions can result in reduced cardiac output and/or elevated intra-cardiac pressures at rest or during stress.

Examples of the present disclosure provide compliance-enhancing band implant devices, which may be implanted in one or more locations in a compromised aorta and/or other vessel(s). For example, FIG. 1C shows example positions of band-type compliance-enhancing implant devices 101 implanted in various areas of an aorta 16b. Embodiments of the present disclosure provide elastic reshaping of a target blood vessel, such as the aorta, in a manner as to produce a volume differential between high- and low-pressure states, thereby mimicking conditions of a stretchy, healthy blood vessel. Implants of the present disclosure provide a mechanism for changing the shape blood vessel from a relatively more-circular shape in higher-pressure conditions to a relatively less-circular shape (e.g., peanut/figure-eight shape) in relatively lower-pressure conditions,

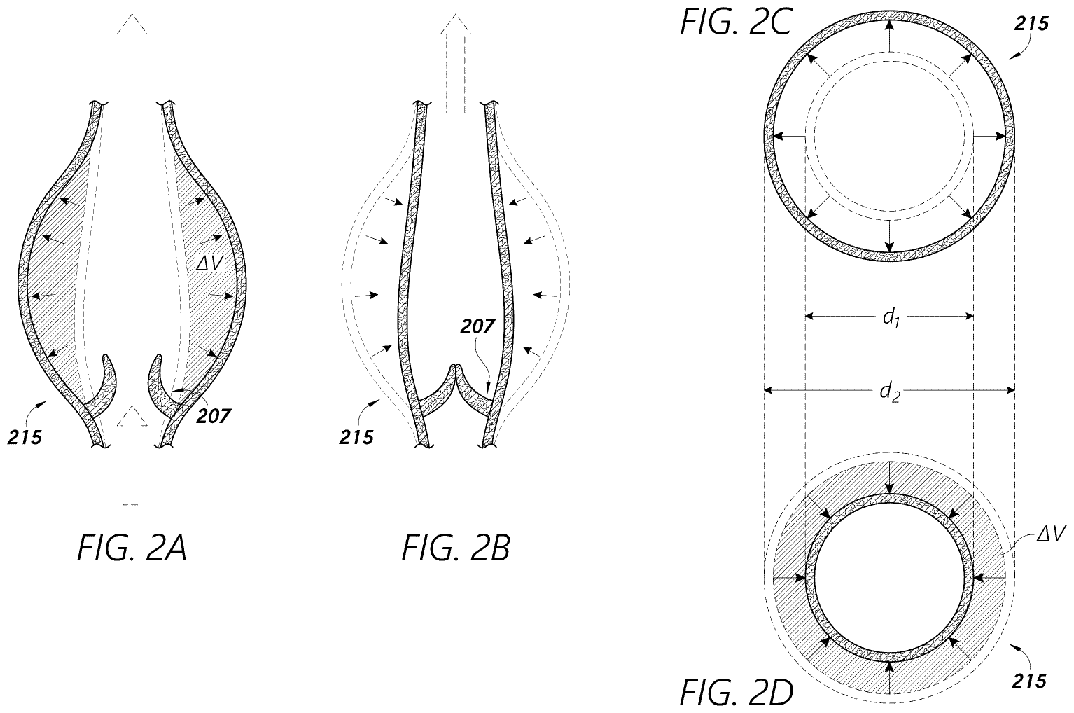

FIGS. 2A and 2C provide side and cross-sectional views, respectively, of a compliant blood vessel 215, such as an artery (e.g., aorta), experiencing expansion during the systolic phase of the cardiac cycle. FIGS. 2B and 2D provide side and cross-sectional views, respectively, of the compliant blood vessel 215 radially contracting/recoiling during the diastolic phase of the cardiac cycle. As understood by those having ordinary skill in the art, the systolic phase of the cardiac cycle is associated with the pumping phase of the left ventricle, while the diastolic phase of the cardiac cycle is associated with the filling phase of the left ventricle. As identified in FIGS. 2A and 2D, with proper arterial compliance, a change in volume ΔV will generally occur in an artery between high- and low-pressure phases of the cardiac cycle. With respect to the aorta, as shown in FIGS. 2A and 2C, as blood is pumped into the aorta 215 through the aortic valve 207, the pressure in the aorta increases and the diameter of at least a portion of the aorta expands. A first portion of the blood entering the aorta 215 during systole may pass through the aorta during the systolic phase, while a second portion (e.g., approximately half of the total blood volume) may be stored in the expanded volume 4V (see FIG. 2A) caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where ΔV is the change in volume (e.g., in mL) of the blood vessel, and ΔP is the pulse pressure from systole to diastole (e.g., in mmHg):

$$C = \frac{Δ V}{Δ P} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Arterial compliance restoration devices, methods, and concepts disclosed herein may be generally described in the context of the ascending aorta. However, it should be understood that such devices, methods and/or concepts may be applicable in connection with any other artery or blood vessel.

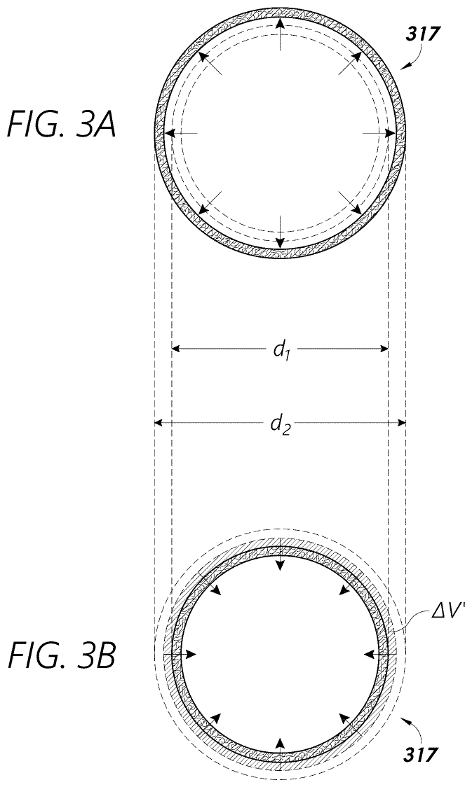

FIGS. 3A and 3B show a cross-sectional profile of a blood vessel 317 that is relatively stiff, similar to the blood vessel 16b shown in FIG. 1C, wherein the compliance of the vessel portion 317 is diminished relative to the healthy aorta 16a as shown in FIG. 1B. Due to the stiffness of the blood vessel wall, the blood vessel 317 may expand a relatively limited amount ΔV′ between diastole (shown in FIG. 3B) and systole (shown in FIG. 3A). That is, during systole, the increased fluid pressure within the blood vessel 317 may result only in a relatively small and/or negligible expansion of the diameter of the blood vessel 317, as shown with respect to the difference between the contracted diameter d₁ and the expanded diameter d₂ in FIGS. 3A and 3B. Due to the limited expansion of the blood vessel 317, the change in volume ΔV′ in the blood vessel between phases of the cardiac cycle may likewise be limited, and therefore relatively little energy is stored in the blood vessel wall in high-pressure conditions and returned to the blood circulation during low-pressure conditions, resulting in more pulsatile blood flow compared to healthy, compliant aortic tissue.

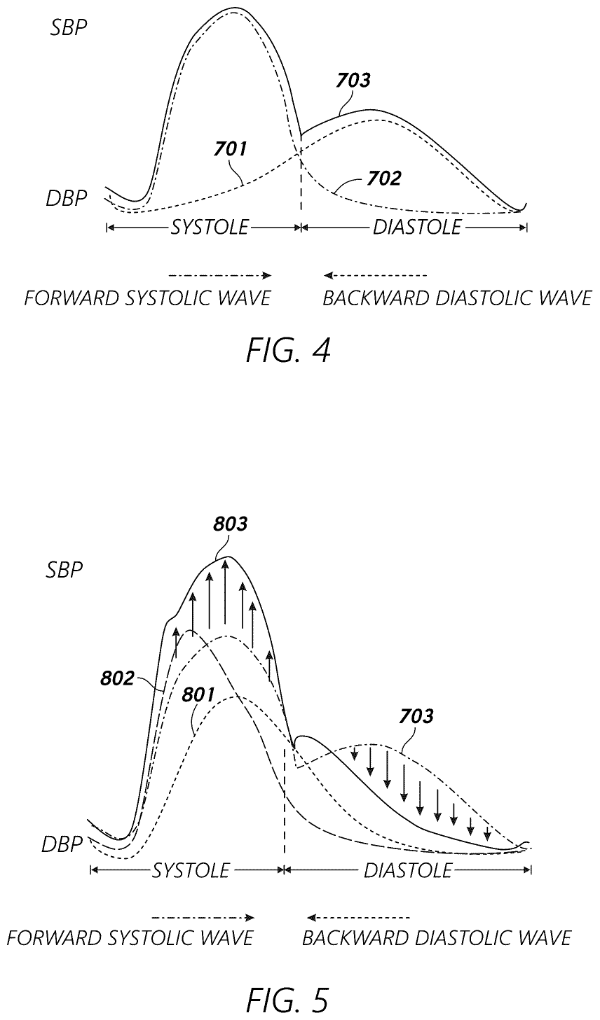

FIG. 4 is a graph illustrating blood pressure over time in an example patient with a healthy, compliant aorta, wherein arterial blood pressure is represented as a combination of a forward systolic pressure wave 702 and a backward diastolic pressure wave 701. The combination of the systolic wave 702 and the diastolic wave 701 are approximately represented by the waveform 703.

FIG. 5 is a graph illustrating blood pressure over time in an example patient having reduced aortic compliance. The graph of FIG. 5 shows, for reference purposes, the example combined wave 703 shown in FIG. 4. When low compliance is exhibited, less energy may be stored in the aorta compared to a healthy patient. Therefore, the systolic waveform 802 may demonstrate increased pressure during the systolic phase relative to a patient having normal compliance, while the diastolic waveform 801 may demonstrate reduced pressure during the diastolic phase relative to a patient having normal compliance. Therefore, the resulting combined waveform 803 may represent an increase in the systolic peak and a drop in the diastolic pressure, which may cause various health complications. For example, the change in waveform may impact the workload on the left ventricle and may adversely affect coronary profusion.

In view of the health complications that may be associated with reduced arterial compliance, as described above, it may be desirable in certain patients and/or under certain conditions, to at least partially alter compliance properties of the aorta or other artery or blood vessel, or otherwise alter/control flow therein, in order to improve cardiac/organ health. Disclosed herein are various devices and methods for at least partially restoring and/or increasing compliance in a blood vessel, such as the aorta. Certain examples disclosed herein achieve restoration of arterial compliance through the use of implantable energy-storing bands configured to be connected across at least a portion of an inner diameter of the aorta or other blood vessel to generate vascular compliance. For example, such bands may be configured to expand in accordance with elastic features/characteristics thereof and store energy during higher-pressure periods of the cardiac cycle (e.g., during the systolic phase). During lower-pressure periods (e.g., during the diastolic phase), such band implant devices contract/shorten to reshape the target blood vessel in a manner as to reduce a volume thereof to thereby return the stored energy to the circulation and increase flow through the vessel.

In some examples, devices of the present disclosure include bands comprising springs or other elastic mechanisms having a shape-memory, such that elongation thereof stores energy in the band due to the biasing of the band towards a shorter, contracted configuration. When anchored across a blood vessel internal diameter/chord, increased transmural blood pressure in the aorta (or other target blood vessel) may cause the elastic band to elongate/expand in the lengthwise dimension. With the band spanning the interior of the target blood vessel, as blood pressure decreases in connection with lower-pressure stages/phases of the cardiac cycle (e.g., diastole), the band is permitted to contract/recoil back to its shorter configuration that more closely approximates the biased length/shape of the band and/or spring element(s) thereof.

Devices of the present disclosure may be implanted in a target blood vessel, such as in the aorta (e.g., aortic trunk, descending thoracic, or abdominal aorta), using transcatheter and/or other minimally-invasive means, such as through a direct minimally-invasive path to the exterior of the aorta through the back and/or flank of the patient. With respect to transcatheter procedures, compliance-enhancement bands of the present disclosure may be advanced to the target area of the blood vessel through the vasculature, wherein each of the band(s) is anchored to two or more circumferential wall segments of the blood vessel from within the blood vessel.

### Compliance-Enhancing Band Implants

The present disclosure relates to systems, devices, and methods for adding back and/or increasing compliance in the aorta or other arterial (or venous) blood vessel(s) to provide improved perfusion of the heart muscle and/or other organ(s) of the body. Examples of the present disclosure can include elastic bands that, when implanted, are configured to decrease the cross-sectional area of the blood vessel segment in which the band(s) is/are implanted during low-pressure conditions, such as diastole, which serves to force blood through the blood vessel segment by pushing the blood through as the vessel volume reduces in connection with band contraction.

The elastic band implant devices of the present disclosure generate a differential cross-sectional area or volume of the target blood vessel(s) (e.g., aorta) between high- and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel.

As the implants of the present disclosure produce complaint blood vessel volume change by manipulating/reshaping the native blood vessel walls, compliance can be increased in the target blood vessel without requiring blood vessel grafting or resection. Therefore, compared to blood flow solutions involving blood vessel grafting/resection, examples of the present disclosure can provide a solution that avoids the risks that may be associated with cutting of the vessel and/or devices grafted in/to such vessels, which may present risk of rupture and blood leakage. Hazards associated with extravascular arterial blood leakage, such as within the abdominal and/or chest cavity, can include the risk of serious injury or death. By implementing relatively small punctures in the blood vessel to deploy band tissue anchors, as opposed to blood vessel resection, risks of blood leakage can be reduced.

As described above, desirable diastolic flow in arterial blood vessels is enabled by the decrease in cross-sectional area/volume of the blood vessels when transitioning from higher-pressure conditions (e.g., systole) to lower-pressure conditions (e.g., diastole). Where the relevant blood vessel has become stiff and non-compliant, stretching/expanding and subsequent contraction/shrinking of the blood vessel to cause the desired change in area/volume of the blood vessel may be limited due to the perimeter/wall of the blood vessel resisting stretching. Examples of the present disclosure provide implants that cause a change in cross-sectional area/volume of a target blood vessel without requiring stretching in the blood vessel wall. Rather, such cyclical change in blood vessel area/volume can be achieved through manipulation of the shape (e.g., cross-sectional shape) of the target blood vessel, wherein a transition between blood vessel shapes occurring in response to changing pressure conditions can reduce and increase the area/volume of the blood vessel in a cyclical manner to promote more even flow of blood through the blood vessel throughout the cardiac cycle.

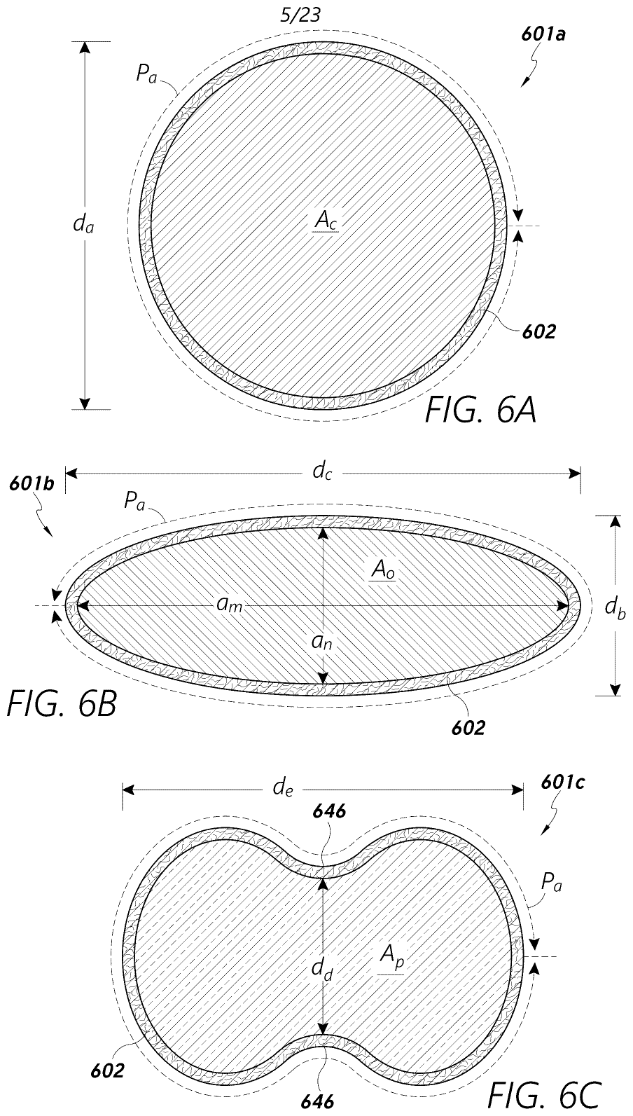

With respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area of the blood vessel. FIG. 6A shows an example blood vessel 601 (identified as blood vessel 601a in FIG. 6A) having a generally circular cross-sectional shape, such that the area A_{c} thereof is maximized for the given perimeter/wall-length Pₐ. In the circular configuration, the diameter dₐ is substantially constant at every angle about the axis of the vessel.

Diverging from a circular cross-sectional shape can produce a cross-sectional area/volume for a blood vessel that is less than the maximum area A_{c} shown in FIG. 6A. For example, FIG. 6B shows the blood vessel 601 (identified as vessel 601b in FIG. 6B) having an oval shape, which produces the cross-sectional area Aₒ that is less than the area A_{c} with the same blood vessel wall/perimeter length Pₐ. The oval shape of the vessel 601b may have a major axis aₘ having a dimension d_{c} that is greater than a dimension d_{b} of the minor axis aₙ thereof.

FIG. 6C shows the blood vessel 601 (identified as vessel 601c in FIG. 6C) in an alternative non-circular shape, wherein opposing wall portions of the blood vessel 601c are brought together to form a “peanut” shape with mid-portions 646 of the vessel wall 602 deflected inward from opposite sides such that the mid-portions 646 may be relatively close together across the vessel lumen/channel. The cross-sectional area Aₚ of the peanut-shaped vessel 601c is similarly smaller than the circular area A_{c} of the vessel 601a. The area Aₚ may further be less than the area Aₒ of the oval-shaped vessel 601b, such that a change in area between the circular shape 601a and the peanut shape 601c may produce a difference in area/volume that is greater than a transition between the circular shape 601a and an oval shape 601b, depending on the shortened dimension d_{d}.

Due to the areas Aₒ/Aₚ being less than the area A_{c} of the circular configuration shown in FIG. 6A, transitioning from the circular shape 601a to the non-circular shapes 601b, 601c can provide a reduction in area/volume of the blood vessel, and therefore solutions that cause transitions between circular and non-circular blood vessel shapes between cardiac phases can provide compliance characteristics without the need for elasticity in the blood vessel wall tissue. For example, where a mechanism is implemented to cause a blood vessel to transition between circular and non-circular shapes in response to changing pressure conditions, such manipulation of the blood vessel shape can introduce volumetric change in the blood vessel in response to the typical changes in pressure experienced during the cardiac cycle, thereby increasing cardiac efficiency and reducing pulsatile load.

In view of the foregoing, examples of the present disclosure provide implant devices and processes configured to transition the shape/area of a blood vessel from circular/more-circular to non-circular/less-circular shapes to enhance compliance with respect to the area of the implant reshaping. Such implant devices/processes may incorporate any suitable or desirable reshaping mechanism configured to produce vessel reshaping in a way that allows for a change in blood vessel area/volume between the systolic and diastolic phases of the cardiac cycle. As described above, for relatively stiff blood vessels, radial outward expansion/stretching of the blood vessel sufficient to achieve a change in volume that produces desirable compliance may not occur as pressure conditions change. Using implant devices in accordance with aspects of the present disclosure may be desirable to provide the necessary change in volume of the target blood vessel.

Examples of the present disclosure provide for band-type implants that are biased to a relatively shorter lengthwise dimension, wherein such bands are configured to elastically stretch/expand in the lengthwise direction as pulled apart by the blood vessel wall portions in which the ends of the band are implanted when pressure within the blood vessel overcomes the bias of the band and causes the blood vessel wall to pull the band to the elongated configuration. Band-type vessel reshaping implants can be preferable compared to stent-type vessel reshaping solutions due to the ability of such bands to be relatively minimalistic compared to certain stent structures. Furthermore, relatively complex reshaping, such as the formation of the peanut-type shapes described above, can be mechanically complex or difficult for a stent to achieve, whereas the band implants presented herein can be well-suited to produce such shapes. The ability of band-type implant devices to reshape the target blood vessel in the manner described above to produce the desired oval- or peanut-shaped cross section of the blood vessel can be achievable due to stiff/non-compliant blood vessels, which may be unable to stretch to a substantial degree, still retaining the ability to bend to a sufficient degree to allow for such shaping of the blood vessel. That is, the bending stiffness of a non-compliant blood vessel may be relatively lower compared to the stretching stiffness thereof. Therefore, examples of the present disclosure achieve compliance through bending energy with respect to the blood vessel wall, as opposed to stretching energy. When band implants in accordance with aspects of the present disclosure are elongated/stretched, energy may be stored therein, wherein recoil/contraction of the band towards its biased, shortened configuration can return/release energy to the blood vessel wall, and thereby to the blood circulation.

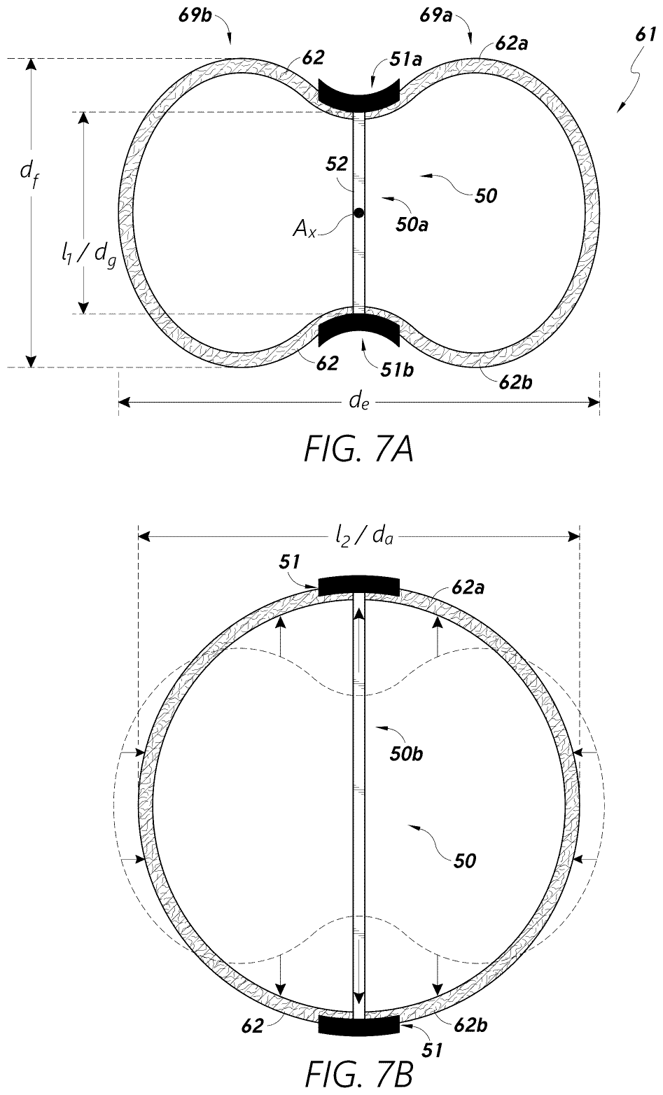

FIGS. 7A and 7B show a compliance-enhancing band implant device 50 implanted in a blood vessel 61 in accordance with examples of the present disclosure. The images of FIGS. 7A and 7B show cross-sectional views of the blood vessel segment 61 in which the implant device 50 is implanted. As with other examples, the band implant 50 is configured to stretch in response to increased luminal pressure in the blood vessel 61 due to anchoring of the band 50 to the blood vessel walls 62, such that high pressure in the blood vessel 61 that causes radial outward force against the blood vessel walls 62 pulls on the respective ends 51 of the band implant 50 to stretch/elongate the band 50. FIG. 7B shows the stretched configuration 50b of the band.

The stretching of the band 50 to the lengthened/elongated configuration 50b shown in FIG. 7B causes energy to be stored in the elasticity of the band 50, such that energy is returned to the blood vessel walls 62, and therefore to the blood circulation within the blood vessel segment, as pressure decreases. The elasticity of the band is configured to be sufficient to overcome the pressure forces within the blood vessel 61 to return the band 50 to the contracted/shortened configuration shown in FIG. 7A in the presence of diastolic pressure conditions, thereby reshaping the blood vessel 61 to a non-circular cross-sectional shape, such as the illustrated peanut/figure-eight shape. With respect to implantation within the aorta or other arterial blood vessel, the systolic phase of the cardiac cycle, during which pressure levels in the aorta/arteries are relatively higher, causes the expansion of the band 50 shown in FIG. 7B, whereas the diastolic phase, which is associated with relatively lower arterial blood pressure levels, causes the axial/longitudinal contraction of the band 50 to return to the shortened, low-energy configuration/state shown in FIG. 7A.

The band 50 may have any suitable or desirable elastic mechanism configured to bias the band to the contracted/shortened state 50a of FIG. 7A. For example, in some implementations, the band 50 comprises one or more spring elements, which may be longitudinally oriented with respect to the orientation of the band 50, wherein the spring element(s) serve as elastic object(s) that, when stretched, exert a restoring force which tends to bring the spring element(s) back to an original/biased length thereof as elongation forces subside. The restoring force of such spring elements may generally be proportional to the amount of stretch/elongation of the spring, as in accordance with Hook's law.

The natural cross-sectional shape of the aorta may generally be circular; as explained above, for a given blood vessel wall circumference/perimeter length, the circular configuration of the blood vessel may provide the maximum area/volume within the respective blood vessel segment. Therefore, any deviation from such circular/cylindrical form of the blood vessel wall may decrease the area/volume within the respective blood vessel segment. With the shortened band 50a anchored to opposing wall portions 62a, 62b of the blood vessel 61, the wall portions 62a, 62b may be pulled at least partially towards an axial center Aₓ of the blood vessel 61 and/or towards each other in a manner as to cause the blood vessel 61 to form a non-circular/-cylindrical shape, such as the peanut-type shape shown in FIG. 7A. That is, compared to the circular/cylindrical shape shown in FIG. 7B, the blood vessel 61 in FIG. 7A has a cross-sectional area in the axial segment/area where the band 50 is implanted that is reduced. The resulting shape of the blood vessel 61 when the band 50a is in the shortened configuration can reduce a diameter/dimension d_{g} of the blood vessel parallel with the orientation of the band 50 in the area where the band 50 is implanted. In some implementations, another dimension/diameter de of the blood vessel 61 (e.g., a dimension orthogonal to the lengthwise dimension of the band 50) may become elongated when the wall portions 62 are brought closer together relative to the diameter dₐ of the circular configuration of FIG. 7B. When the band 50 is contracted, as shown in FIG. 7A, the blood vessel 61 may form bulging/bulbous portions 69a, 69b on either side of the band 50a, wherein such portions have a diameter/dimension d_{f} that is greater than the shortened diameter d_{g} in the area between the anchors 51 of the implant device 50.

As with any example disclosed herein including elastic band feature(s), such features can comprise any suitable or desirable elastic/stretchable material. Example materials and structures of elastic-band-type components (e.g., flexure, torsional, and/or coil elements/features) disclosed herein include superelastic nitinol, high-strength CoCr alloys, stainless steel (e.g., 316LVM), and the like.

The band 50 may be anchored to the blood vessel 61 in any suitable or desirable manner. For example, in some implementations, tissue anchors 51 are associated with each lengthwise end of the band 50, as shown. As with any tissue anchors disclosed herein, the anchors 51 may comprise patches, pledgets, pins, coils, screws, tabs, hooks, or other retention member/means configured to embed-in and/or hold ends of the band 50 to/against the respective wall portions 62 of the blood vessel 61. In some implementations, the tissue anchors 51 may be disposed primarily on the outer diameter of the blood vessel 61, such that the anchors 51 are disposed at least partially externally of the blood vessel 61. The band portion 52 of the implant 50 can be disposed primarily within the blood vessel 61. Portion(s) of the band portion 52 and/or anchors 51 may pass through the blood vessel wall portions 62.

The anchors 51 can each include a distal tissue-engagement feature/structure, wherein the term ‘distal’ in this context refers to being outwardly positioned with respect to a proximal lengthwise center of the band 50 (at the axis Aₓ of the blood vessel in the images of FIGS. 7A and 7B). Such tissue-engagement feature can have a structure that is relatively wide/broad relative to the puncture through the vessel wall, such that the anchor 51 provides interference preventing the anchor from being pulled back through the vessel wall puncture/hole. In some examples, the tissue anchors 51 further comprise a proximal (with respect to the tissue-engagement feature) band-engagement feature configured to couple the anchor 51 to the band portion 52 of the implant 50. The anchors 51, as shown, can be deployed in, on, and/or through the tissue walls 62.

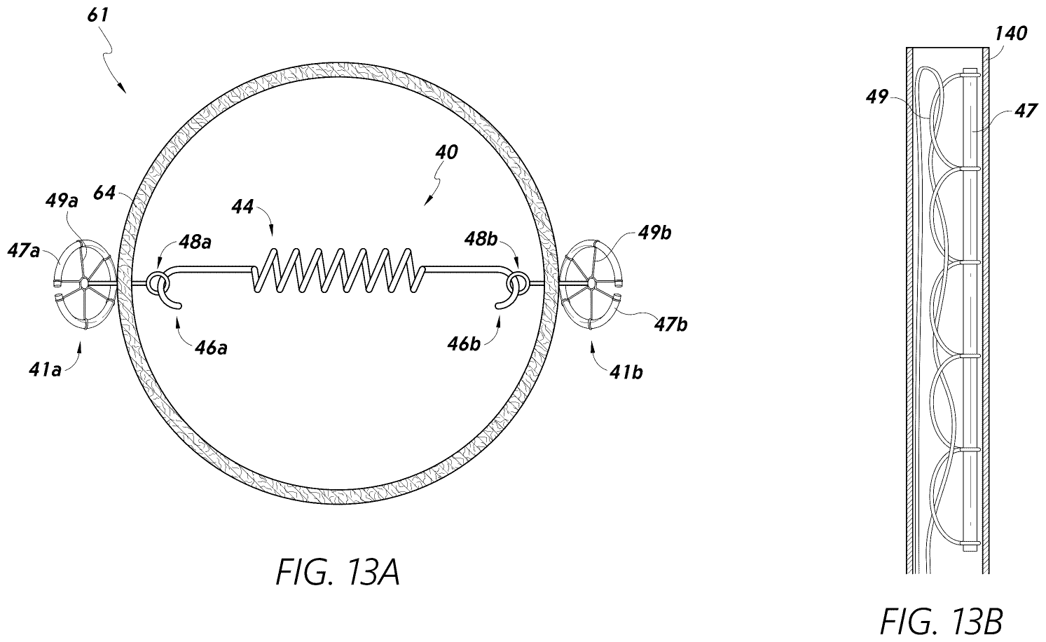

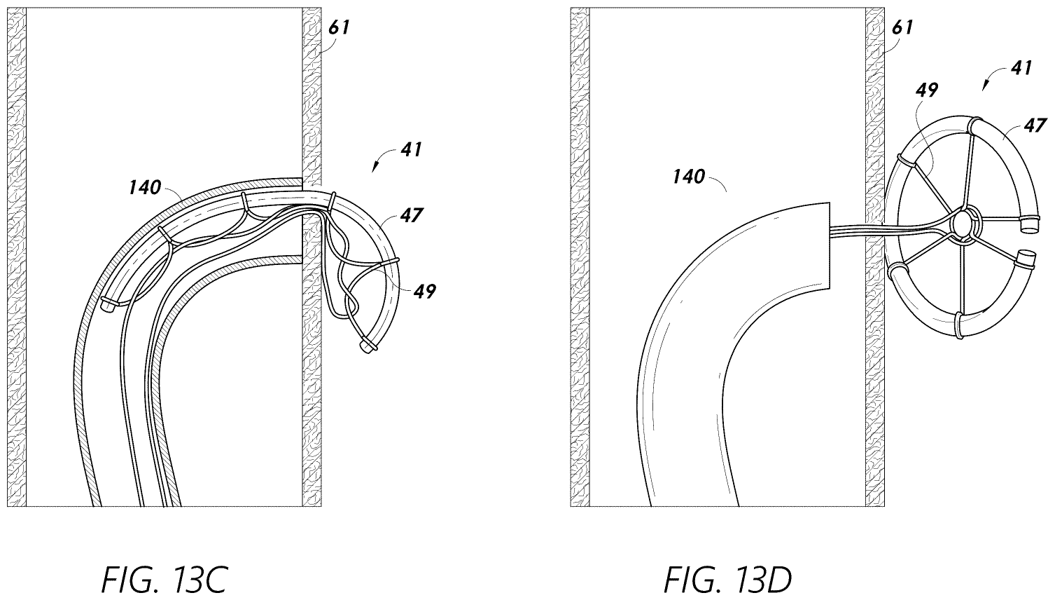

As with any example disclosed herein including tissue anchor feature(s), such features can comprise any suitable or desirable material or structure. Example materials and structures of tissue anchor components (e.g., circular structures as in FIG. 13A) disclosed herein include superelastic nitinol, which may advantageously provide/facilitate a desirably low-profile form for low-profile implantation and/or delivery.

As the pressure in the blood vessel 61 increases (e.g., in connection with the systolic phase of the cardiac cycle), the elastic nature of the band 50 allows for the ends 51 of the band 50 to be pulled to an elongated configuration, wherein the band portion 52 is axially/longitudinally stretched/expanded and stores energy based on a bias of the band 52 towards the contracted/shortened state 50a. The band may comprise any elastic structure or material, such as a spring or elastic polymer strip. When stretched/expanded, the band 50 allows the blood vessel 61 to assume a more circular cross-sectional shape, as shown in FIG. 7B. That is, the band 50 improves cardiac perfusion by causing a decrease in cross-sectional blood vessel area during diastole relative to systole due to the reshaping of the blood vessel caused by the contraction of the band 50. For example, for an insufficiently-compliant blood vessel, the outer wall/perimeter of the blood vessel may generally not change sufficiently between systole in diastole due to a lack of stretching of the blood vessel wall. Therefore, with a typical circular cross-sectional shape throughout the cardiac cycle, the cross-sectional area of the blood vessel may not change sufficiently between cardiac phases, resulting in poor perfusion. Examples of the present disclosure solve this problem by leveraging the principle that a non-circular cross-sectional shape will have lesser area than a circular cross-sectional shape having the same perimeter length. In particular, implant devices presented herein are configured to achieve improved perfusion by changing the cross-sectional area of the aorta (or other blood vessel) from a more-circular to a less-circular shape as the cardiac cycle transitions from systole to diastole. As shown in FIG. 7B, the compliance-enhancing band 50 is stretched in response to higher-pressure conditions to assume a more circular shape for the vessel 61, after which the spring/elastic bias in the band 50 pulls the vessel walls 62 back together as pressure drops, thereby pushing blood through the vessel and in proving perfusion. The band 50 is biased to the longitudinally-contracted state shown in FIG. 7A.

The tissue anchors 51 may comprise distal tissue-engagement features for puncturing and/or holding the tissue wall. Furthermore, the anchors 51 may include proximal band-engagement features for coupling to the band portion 52 of the device 50. Such band-engagement means may comprise one or more loops, hook, clips, tabs, or the like. With respect to tissue anchors described herein, the terms “anchored in,” “anchored on,” “anchored to,” and “anchored through” a tissue wall, such terms are used according to their broad and ordinary meanings, and may describe an anchor element contacting, holding, embedding-in, puncturing-through, adhering-to, or otherwise engaging with, a tissue wall. The recited terms may be used interchangeably in some contexts herein.

With respect to band devices of the present disclosure that are implanted by passing the band and/or anchors associated therewith through the blood vessel wall to anchor to the blood vessel on the outside of the blood vessel wall, it may generally be necessary to implement puncture holes through the blood vessel wall for such purpose. However, the presence of such punctures/holes in the blood vessel wall, particularly with respect to the relatively high-pressure aorta, can present risks of hemorrhaging. Furthermore, over time and over the course of many cardiac cycles, such holes can become eroded or enlarged, further increasing the risks of leakage. Therefore, it may be desirable to implement certain mechanisms to reduce risks of injury to the patient with respect to arterial leakage/rupture.

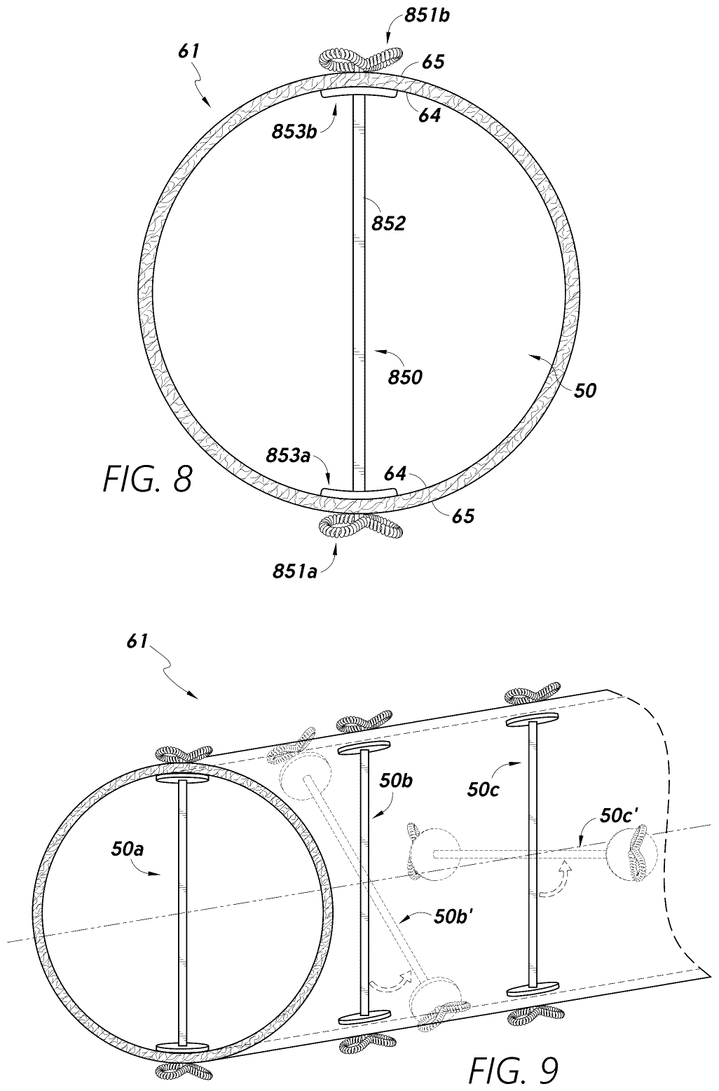

FIG. 8 shows a compliance-enhancing band implant 850 implanted in a blood vessel 61, wherein tissue anchors 851 are associated with longitudinal/lengthwise ends of the implant device 850. The tissue anchors 851 may be configured to be deployed on an exterior/outer diameter/surface of the blood vessel 61, wherein the anchors 851 may pull-against/contact the exterior/outer wall 65 of the blood vessel 61. In addition, the band implant device 850 may have interior hemostasis elements 853, which may be disposed against the inner wall/diameter/surface 64 of the blood vessel 61, such that the tissue anchors 851 and the hemostasis elements 853 sandwich the blood vessel wall 62 therebetween.

The band portion 852 of the implant 850 may pass through and/or otherwise engage with the hemostasis elements 853a, 853b. In some implementations, the hemostasis elements 853 are secured to the band portion 852 in a manner as to hold the hemostasis elements 853 in-place and against the inner wall 64. The tissue anchors 851 and/or hemostasis elements 853 may comprise patches, pledgets, or similar forms/structures, wherein such forms may advantageously provide fluid-sealing characteristics. The hemostasis elements 853 may comprise pledgets, patches, sheets, plugs, or any other form or structure configured to improve the fluid seal around the band portion 852 (or tissue anchor portion) passing through the tissue wall 62. In some implementations, the hemostasis elements 853 comprise foam forms, such as round/circular foam pieces, wherein the band portion 852 passes through the foam. Although knot-type anchors 851 are shown, other types of anchors may be implemented. For example, in some implementations, both the anchors 851 and hemostasis elements 853 comprise fluid-sealing patches/pledgets.

The hemostasis elements 853 may provide both sealing and anchoring functionality for the implant device 850. For example, the hemostasis elements 853 may provide further securement/attachment to the tissue wall 64. In some implementations, the hemostasis elements 853 may comprise anchoring features, such as barbs, pins, or the like, which may be configured to embed in the tissue wall 64. Furthermore, tissue ingrowth after implantation may further secure the hemostasis elements 853 to the inner wall 64 of the blood vessel 61. In some implementations, once the tissue anchors 851 have been deployed and the hemostasis elements 853 disposed within the blood vessel 61, the internal blood pressure within the blood vessel 61 may serve to exert outward radial force against the hemostasis elements 853 and against the vessel wall 64 in a manner as to hold the hemostasis elements 853 in a sealing engagement against the inner wall 64, thereby improving the seal around the hole/puncture through the tissue wall.

In some implementations, as shown in FIG. 8, the tissue anchors 851 may comprise bulky knot forms/structures, which may comprise line(s) of suture wound and/or looped in a configuration as to provide a tissue-anchoring form. Examples of such knot-type tissue anchors that can be used in connection with band-type compliance-enhancement implant devices of the present disclosure are disclosed in Int'l Pub. WO 2013/003228, entitled “Transapical Mitral Valve Repair Device,” filed on Jun. 22, 2012, the disclosure of which is hereby incorporated by reference in its entirety.

In some implementations, multiple compliance-enhancing band implants in accordance with aspects of the present disclosure may be implanted in a single vascular system, blood vessel, or blood vessel segment. FIG. 9 shows a blood vessel 61 having multiple compliance-enhancing implant devices 50 in accordance with the present disclosure implanted therein in an axially-offset arrangement. The band implants 50a, 50b, 50c may be implanted in close proximity to one another, or may be offset by any suitable or desirable distance within the blood vessel with respect to the longitudinal/axial dimension of the blood vessel. Although three implant devices 50a, 50b, 50c are shown, it should be understood that any number of band devices may be implanted in a given vasculature, blood vessel, or segment thereof, such as two implant devices.

The multiple implant devices 50 may have a common rotational orientation/alignment with respect to the axis of the blood vessel 61, as shown in solid line representations 50a, 50b, 50c of the various bands having common rotational orientation. Alternatively, adjacent bands may be rotationally offset, as represented by the dashed representations of the bands 50a′, 50b′, 50c′. Where the multiple bands are implanted in rotated relative orientations (as shown in dashed-line), elastic contraction of the various bands may further provide a twisting/torsional effect on the blood vessel 61, which may further enhance blood flow through the blood vessel. Furthermore, rotational/angular offsetting of the bands may produce a spiraling/helical internal volume/form through the blood vessel when the bands are contracted, which may produce desirable fluid dynamics, such as by channeling fluid in a manner as to facilitate improved throughput of fluid through the blood vessel.

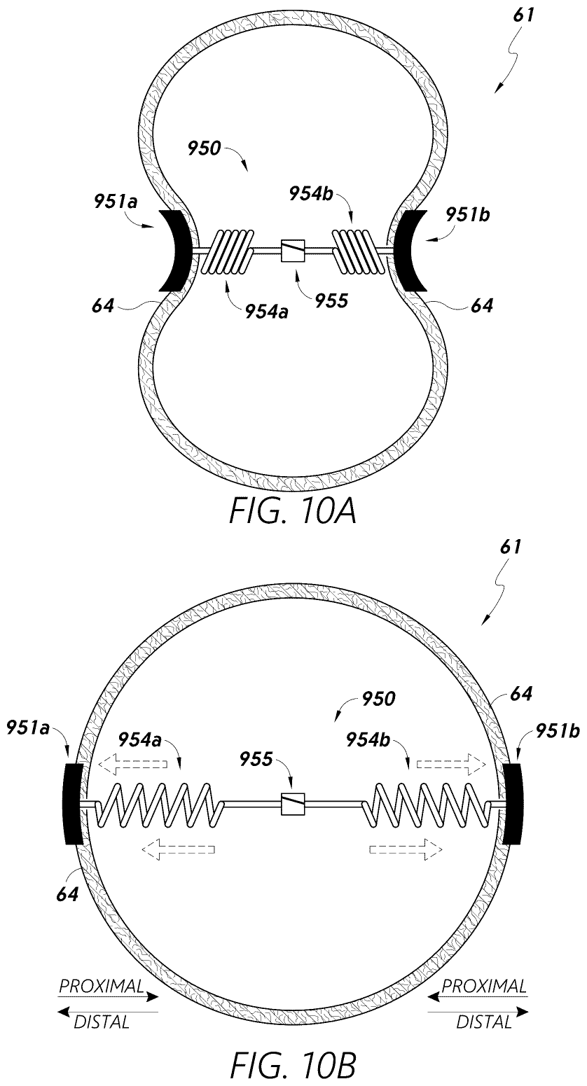

In some implementations, a compliance-enhancing band implant device in accordance with aspects of the present disclosure can be implemented in multiple segments that are coupled together. Such coupling may be performed prior to implantation of the device, or within the target blood vessel during implantation. FIGS. 10A and 10B show an implant device 950 comprising first 954a and second 954b segments that are joined by a lock/fixation element 955 at a center (or other area) of the band 950. Each of the band segments 954a, 954b has associated with an end portion thereof a respective tissue anchor 951, which may be configured to anchor to the wall 64 of the blood vessel 61 on an exterior and/or interior surface thereof. The lock/fixation element 955, as with any other connecting elements/components disclosed herein, can comprise any suitable or desirable material or structure. Example materials and structures of band-connecting components disclosed herein include flexible multi-filament sutures or synthetic fabric.

Each of the band segments 954a, 954b may comprise an elastic element configured to elongate and contract in response to pulling forces, and the removal thereof. Such clastic element(s) can comprise a spring or other stretchable material or structure having biasing or shape-memory biased towards a shortened configuration thereof. The locking element 955 may have any suitable or desirable configuration, such as one or more clamps, clips, hooks, loops, knots, apertures, grooves, traps, or the like.

Implantation of the band device 950 may comprise accessing the target area of the blood vessel transvascularly, deploying the anchors 951 separately, and subsequently coupling the proximal ends/portions of the respective band segments 954 to one another using the locking means/element 955. Some implementations may allow for cinching/tensioning of the band segments 954 prior to locking them together to provide desirable tension for the band implant 950.

The springs/segments 954 of the device 950 may be biased in the contracted state, such that the band 950 is inclined to assume the contracted configuration shown in FIG. 10A in the absence of sufficient fluid pressure pushing outward against the inner diameter of the blood vessel 61 to overcome the biasing of the springs/segments 954 and causing the segments 954 to elongate/stretch-out, as shown in FIG. 10B. Although springs are shown in the examples of FIGS. 10A and 10B, it should be understood that other stretchable elements may be implemented additionally or as alternatives, including elastically-stretchable strings, ropes, lines, cables, or the like.

Although some examples are disclosed herein in which a compliance-enhancing band implant device is implanted in a manner as to bisect the blood vessel cylinder by passing substantially across the center/axis of the blood vessel from opposite circumferential sides of the blood vessel, it should be understood that compliance band implant devices in accordance with aspects of the present disclosure can be anchored at any point along the circumference of the vessel. For example, band implant devices of the present disclosure may be implanted in a manner such that the band portion thereof passes/cuts across the vessel interior along a path/chord that does not overlap or pass in close proximity to the axis of the blood vessel, but rather passes in an outer area of the blood vessel interior. Furthermore, although certain implant devices are disclosed herein that comprise two tissue anchors, it should be understood that any example of the present disclosure may include more than two tissue anchors, such as three or four tissue anchors, wherein the tissue anchors may be coupled in some manner as to cause a pulling/contraction to pull the tissue anchors inward and/or towards one another.

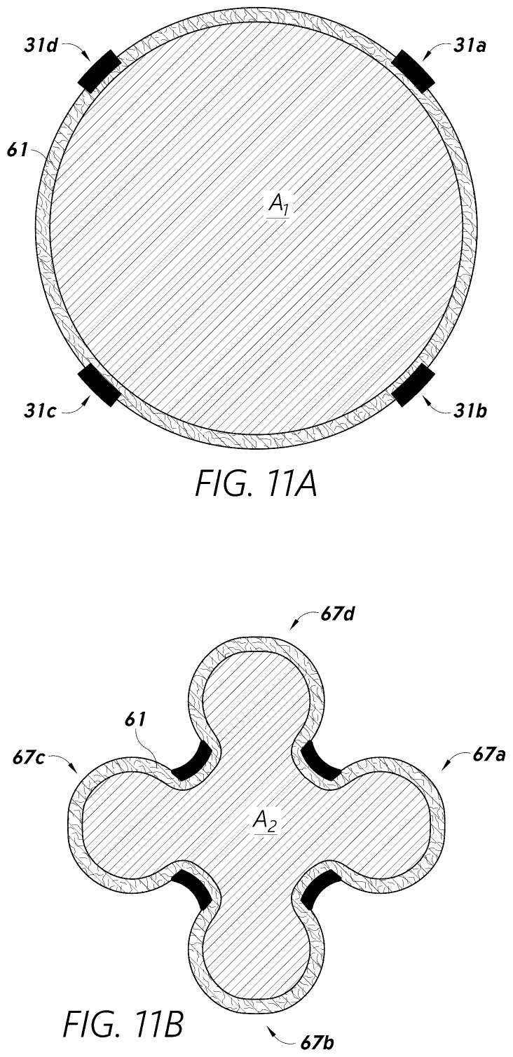

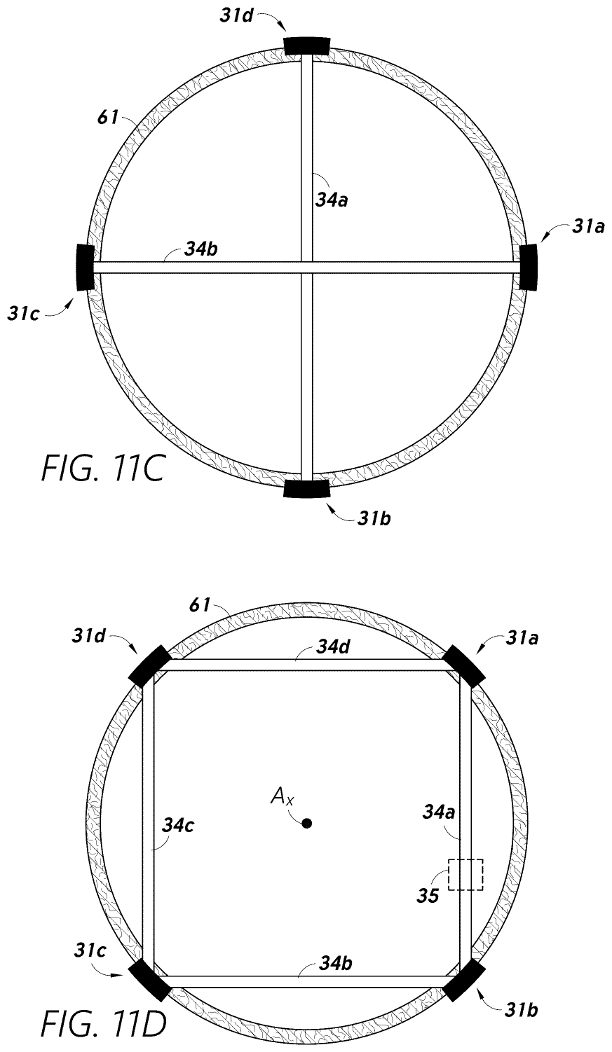

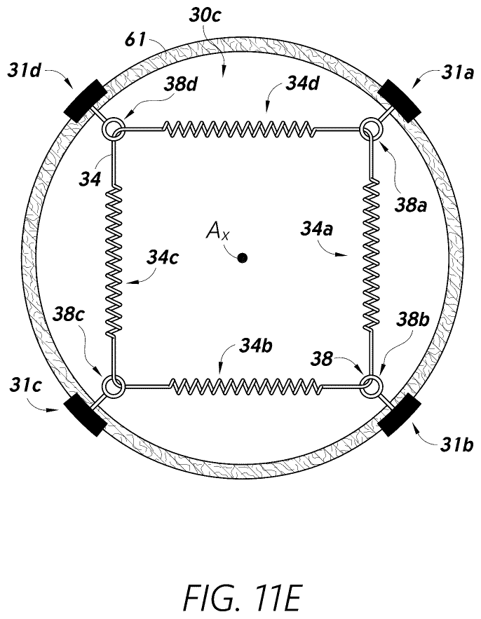

FIGS. 11A, 11B, 11C, 11D, and 11E illustrate example configurations of band-type compliance-enhancing implant devices comprising four tissue anchors. Use of four tissue anchors (or three tissue anchors) may be desirable as an alternative to two-anchor solutions for various reasons. For example, with respect to two-anchor compliance-enhancing implant devices of the present disclosure, a single diametrical bifurcation of the blood vessel by the implanted band device, wherein the band portion of the device passes generally across the center/axis of the blood vessel, can cause the band portion to interfere with the blood flow through the center of the blood vessel in an undesirable way/degree. Furthermore, when implanting a two-anchor implant device across a blood vessel, inexact placement/implantation of the anchors thereof such that the band portion of the device does not pass exactly along the diametrical center of the blood vessel can result in uneven pulling against the blood vessel walls in a manner as to promote migration/cutting of the band portion through the tissue wall in the area where the anchors are implanted. For example, misaligned deployment/implantation of tissue anchors of a two-anchor band device can cause biasing to one diametrical side of the blood vessel cross-section, such that the distribution of forces on the band and anchors associated therewith may tend to push the band away from the axis/center of the blood vessel, potentially causing cutting or damage to the blood vessel due to migration of the tissue anchors and/or band over time.

FIG. 11A shows four tissue anchors 31 distributed about a perimeter/circumference of a blood vessel portion 61. With four tissue anchors 31 deployed on the blood vessel wall 61, inward pulling/contraction of the tissue anchors 31 may result in a shape having a clover and/or compass/diamond star shape (e.g., rounded isotoxal square star shape), as shown if FIG. 11B. As shown in FIG. 11B, the shape formed by the inward cinching of four tissue anchors can form four projections 67a-67d, wherein the internal area A₂ of the blood vessel in such configuration/shape is less than the area A₁ in the circular/cylindrical form shown in FIG. 11A.

The four anchors 31 can be internally coupled in any suitable or desirable manner to produce a contracted configuration that reduces the internal area/volume of the blood vessel. In the example of FIG. 11C, tissue anchor pairs 31a/31c, 31b/31d positioned opposite one another are coupled across the blood vessel in an ‘x’ configuration, wherein the band segments 34a, 34b coupling respective pairs of the tissue anchors 31 may or may not be coupled to one another (e.g., coupled at a lengthwise center of the bands 34). That is, the example of FIG. 11C may provide two band implants each including a respective band segment 34 coupling two tissue anchors to one another, wherein the implants may be implanted in a common axial position of the blood vessel 61. Alternatively, the band segments 34a, 34b may be coupled to one another, such that the tissue anchors 31 and band segments 34 together comprise a unitary implant device.

In FIG. 11D, adjacent tissue anchors 31 (e.g., anchors 31a, 31b) may be coupled by one of a plurality of band segments 34, such that the band segments 34 are generally positioned around a perimeter of the interior of the blood vessel 61, rather than spanning across the center/axis Aₓ of the blood vessel. The embodiment of FIG. 11D may be desirable due to the band segments 34 not obstructing the center Aₓ of the blood vessel lumen, and therefore the arrangement of FIG. 11D may provide less interference with fluid flow through the blood vessel 61 compared to the embodiment of FIG. 11C. Each of the band segments 34a-34d may comprise a separate band coupled between adjacent anchors 31, or a portion of a single band that is coupled to all of the tissue anchors 31. The band 34 and/or segments thereof may be fixed to the respective anchors 31, or may be slidingly coupled thereto in a manner to allow for the band 34 to longitudinally reorient/correct in a manner as to distribute tension evenly and/or proportionally between/among the tissue anchors 31. The configuration of FIGS. 11D (and 11E) may be considered a series arrangement of the band segments.

FIG. 11E provides another example implementation of a four-anchor implant device 350 that includes band segments 34 coupled between adjacent tissue anchors 31. In the example of FIG. 11E, the tissue anchors 31 include internal band-engagement features 38 to which the band segment(s) 34 is/are coupled. In some implementations, the band-engagement features/means 38 comprise loops, eyelets, or fasteners through which the band 34 may pass and/or slide. In some implementations, the band 34 comprises a single band that forms a loop that passes through all of the engagement features 38 in an at least partially free/sliding configuration, such that the band 34 can longitudinally/axially move, slide, and/or migrate through the engagement features 38 to some degree after implantation and/or in connection with cycling of the heart and/or distribution of tension in the band 34. Such slidable engagement with the engagement features (e.g., eyelet fasteners) 38 may allow the band to self-correct from imprecise tissue anchor placement, such that a greater length of the band 34 may be positionable between adjacent tissue anchors that are positioned a greater distance apart relative to other adjacent tissue anchors, which may prevent unbalanced pulling against tissue anchors that are positioned relatively farther apart than other tissue anchor pairs. For example, where a first pair of the tissue anchors 31 (e.g., anchors 31a, 31b) are a first distance apart, and a second pair of the tissue anchors 31 (e.g., anchors 31b, 31c) are a second distance apart that is greater than the first distance (in such configuration, a third distance between anchors 31c and 31d or between the anchors 31d and 31a may be less than the first distance), a greater portion of the overall length of the band 34 may become positioned/disposed between the second pair of anchors to distribute the tension in the line and prevent over-tensioning of the second pair of anchors towards one another. Therefore, the configuration of FIG. 11E may provide a reduced risk of damage to the blood vessel wall 61 that may be associated with uneven pulling/tensioning on tissue anchors.

The band 34 may comprise one or more spring elements or other elastic elements configured to bias the band 34 to a shortened configuration that pulls the tissue anchors 31 radially inward with respect to the axis Aₓ of the blood vessel 61. Furthermore, the band 34 may include two ends that are coupled together to form a loop, such as using a locking or tying means 35, shown in FIG. 11D.

With respect to any of the examples of FIGS. 11A-11E, implantation of the respective band implant devices may be performed minimally invasively (e.g., through the chest, flank, back, etc.) and/or using a transcatheter procedure involving catheter advancement through the vasculature. With a transcatheter approach, the anchors 31 can be placed on the vessel walls, such as using either a transcatheter or minimally-invasive procedure/technique, after which the band/elastic elements/segments 34 may be placed and locked or coupled in some manner to the tissue anchors 31 and/or to each other using transcatheter instrumentation.

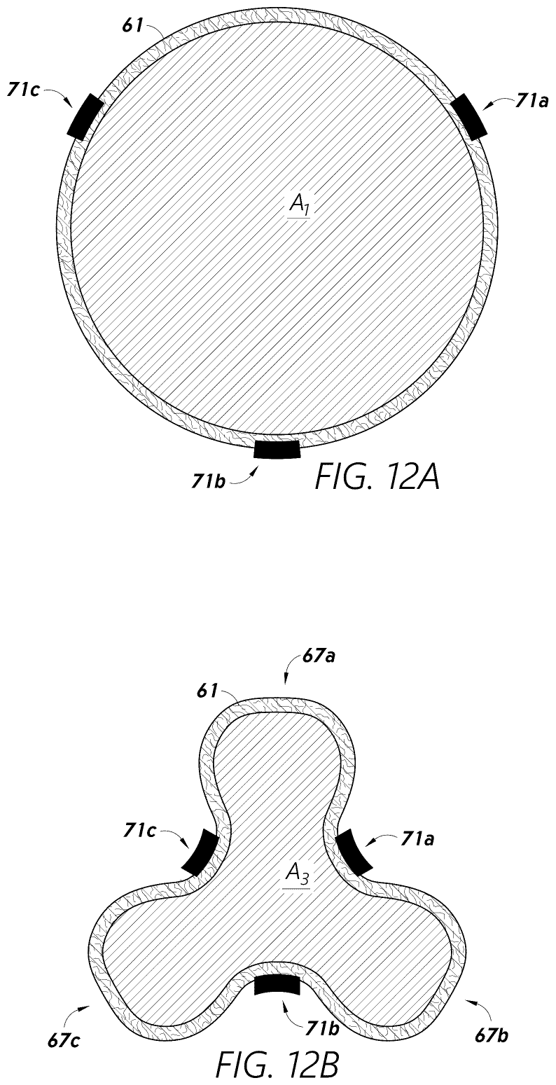

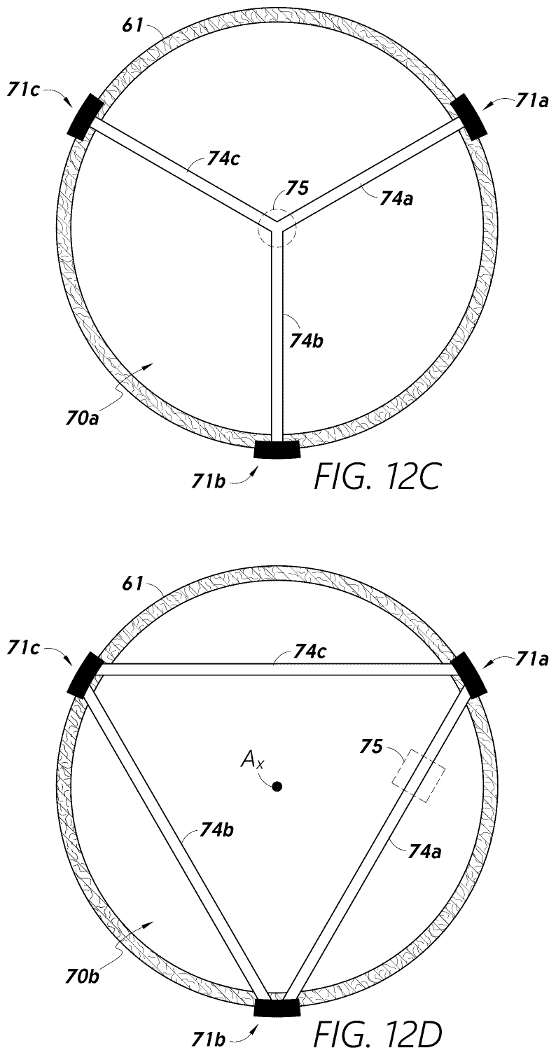

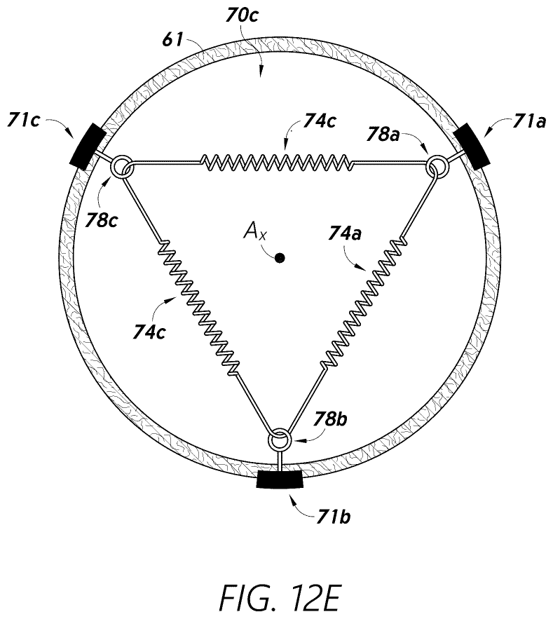

FIGS. 12A, 12B, 12C, 12D, and 12E illustrate example configurations of band-type compliance-enhancing implant devices comprising three tissue anchors, FIG. 12A shows three tissue anchors 71 distributed about a perimeter/circumference of a blood vessel portion 61. With three tissue 71 anchors deployed on the blood vessel wall 61, inward pulling/contraction of the tissue anchors 71 may result in a shape having a triangular star/clover shape (e.g., rounded isotoxal triangle star shape), as shown if FIG. 12B. As shown in FIG. 12B, the shape formed by the inward cinching of three tissue anchors can form three projections 67a-67c, wherein the internal area A₃ of the blood vessel in such configuration is less than the area A₁ in the circular/cylindrical form shown in FIG. 12A.

The three anchors 71 can be internally coupled in any suitable or desirable manner to produce a contracted configuration that reduces the internal area/volume of the blood vessel. In the example of FIG. 12C, the tissue anchors 71 are coupled together in a central area within the blood vessel 61. That is, the example of FIG. 12C may provide three band segments 74a-74c emanating from, and/or coupled to, respective ones of the tissue anchors 71 and coupled together to form a unitary implant device 70a. The three tissue anchors 71 may advantageously be implanted in a common axial position of the blood vessel 61.

In FIG. 12D, adjacent tissue anchors 71 (e.g., anchors 71a, 71b) may be coupled by one 74a of a plurality of band segments 74, such that the band segments 74 are generally positioned around a perimeter of the interior of the blood vessel 61, rather than spanning to/across the center/axis Aₓ of the blood vessel. The embodiment of FIG. 12D may be desirable due to the band segments 74 not obstructing the center Aₓ of the blood vessel lumen, and therefore the arrangement of FIG. 12D may provide less interference with fluid flow through the blood vessel 61 compared to the embodiment of FIG. 12C. Each of the band segments 74a-74c may comprise a separate band coupled between adjacent anchors 71, or a portion of a single band that is coupled to all of the tissue anchors 71. The band 74 and/or segments thereof may be fixed to the respective anchors 71, or may be slidingly coupled thereto in a manner to allow for the band 74 to longitudinally reorient/correct in a manner as to distribute tension evenly and/or proportionally between/among the tissue anchors 71. The configuration of FIGS. 12D (and 12E) may be considered a series arrangement of the band segments, unlike the parallel arrangement of band segments shown in FIG. 12C.

FIG. 12E provides another example implementation of a three-anchor implant device 70c that includes band segments 74 coupled between adjacent tissue anchors 71. In the example of FIG. 12E, the tissue anchors 71 include internal band-engagement features 78 to which the band segment(s) 74 is/are coupled. In some implementations, the band-engagement features/means 78 comprise loops, eyelets, or the like, through which the band 74 may pass and/or slide. In some implementations, the band 74 comprises a single band/line that passes through all of the engagement features 78 in an at least partially free/sliding configuration, such that the band 74 can longitudinally/axially move, slide, and/or migrate through the engagement features 78 to some degree after implantation and/or in connection with cycling of the heart and/or distribution of tension in the band 74. Such slidable engagement with the engagement features 78 may allow the band to self-correct from imprecise tissue anchor placement, such that a greater length of the band 74 may be positionable between adjacent tissue anchors that are positioned a greater distance apart relative to other adjacent tissue anchors, which may prevent unbalanced pulling against tissue anchors that are positioned relatively farther apart than other tissue anchor pairs. For example, where a first pair of the tissue anchors 71 (e.g., anchors 71a, 71b) are a first distance apart, and a second pair of the tissue anchors 71 (e.g., anchors 71b, 71c) are a second distance apart that is greater than the first distance (in such configuration, a third distance between anchors 71c and 71a may be less than the first distance), a greater portion of the overall length of the band 74 may become positioned/disposed between the second pair of anchors through self-correction to distribute the tension in the line and prevent over-tensioning the second pair of anchors towards one another. Therefore, the configuration of FIG. 12E may provide a reduced risk of damage to the blood vessel wall 61 that may be associated with uneven pulling/tensioning on tissue anchors.

The band 74 may comprise one or more spring elements or other elastic elements configured to bias the band 74 to a shortened configuration that pulls the tissue anchors 71 radially inward with respect to the axis Aₓ of the blood vessel 61. Furthermore, the band 74 may include two ends that are coupled together to form a loop, such as using a locking or tying means 75, shown in FIG. 12D.

With respect to any of the examples of FIGS. 12A-12E, implantation of the respective band implant devices may be performed minimally invasively (e.g., through the chest, flank, back, etc.) and/or using a transcatheter procedure involving catheter advancement through the vasculature. With a transcatheter approach, the anchors 71 can be placed on the vessel walls, such as using either a transcatheter or minimally-invasive procedure/technique, after which the band/elastic elements/segments 74 may be placed and locked or coupled in some manner to the tissue anchors 71 and/or to each other using transcatheter instrumentation.

FIGS. 13A-13D illustrates a compliance-enhancing band-type implant device 40 having wheel-and-spoke tissue anchors 41 in various configurations. The implant device 40 includes first 41a and second 41b wheel anchors, wherein the anchors include band-engagement features 48. Although two anchors 41 are shown, it should be understood that the implant device 40 may have any suitable or desirable number of anchors, as described above. The implant device 40 further includes an elastic band segment 44, which may comprise one or more spring or other elastic, energy-storing structure or feature biased to a shortened configuration thereof and configured to stretch/elongate in response to pulling forces exerted on the band 44 by the anchor elements 48.

The band 44 may comprise hook-type engagement features 46, or other types of engagement features, configured to be engaged/coupled with the band-engagement features 48 of the anchors 41. For example, the anchor features 48 may comprise loops, eyelets, hooks, or the like configured to be engaged with by corresponding hooks or other mating features 46 of the band 44. In some implementations, the anchors 41 may be implanted in the blood vessel wall 64, after which the band component 44 may be coupled to the engagement features 48 of the respective anchors 41.

The anchors 41 may have any shape or form as described or referenced herein. In some examples, as illustrated, the anchors 41 may comprise a relatively rigid ring 47, which may be reinforced with sutures or other covering, wherein the ring 47 is held together by spoke connections 49, which may comprise sutures or the like. The spokes 49 may hold the ring 47 in a circular configuration in a wagon-wheel form. The ring 47 may comprise a wire or other bendable material formed into a ring/hoop shape. In some examples, the band-engaging features 48 may comprise suture loops or ties configured to be tied to hoop, hook, or loop structures of the anchor-engagement features 46.

The wheel anchors 41 may be transported to the implantation site in an elongated configuration to allow for relatively low-profile delivery catheter/tools to be used for transcatheter deployment. For example, as shown in FIG. 13B, the ring portion 47 of the anchors 41 may be straightened-out/elongated within a delivery catheter/sheath 140 during delivery. As shown in FIG. 13C, as the tissue anchor 41 is deployed from the delivery system 140, the shape-memory of the ring 47 may assume a curved, circular configuration. In the image of FIG. 13D, the tissue anchor 41 is fully deployed from the delivery system and forms a circular disk, which provides desirable retention structure/surface for holding the tissue anchor against the outer diameter/surface of the target blood vessel when pulled towards the vessel interior.

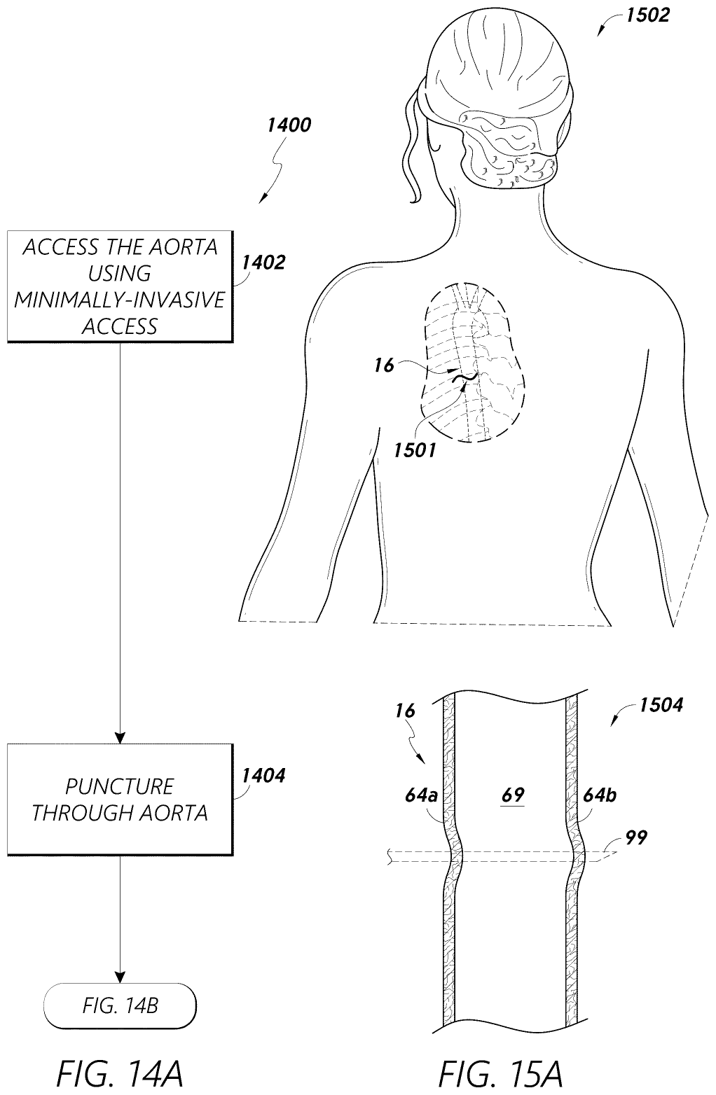

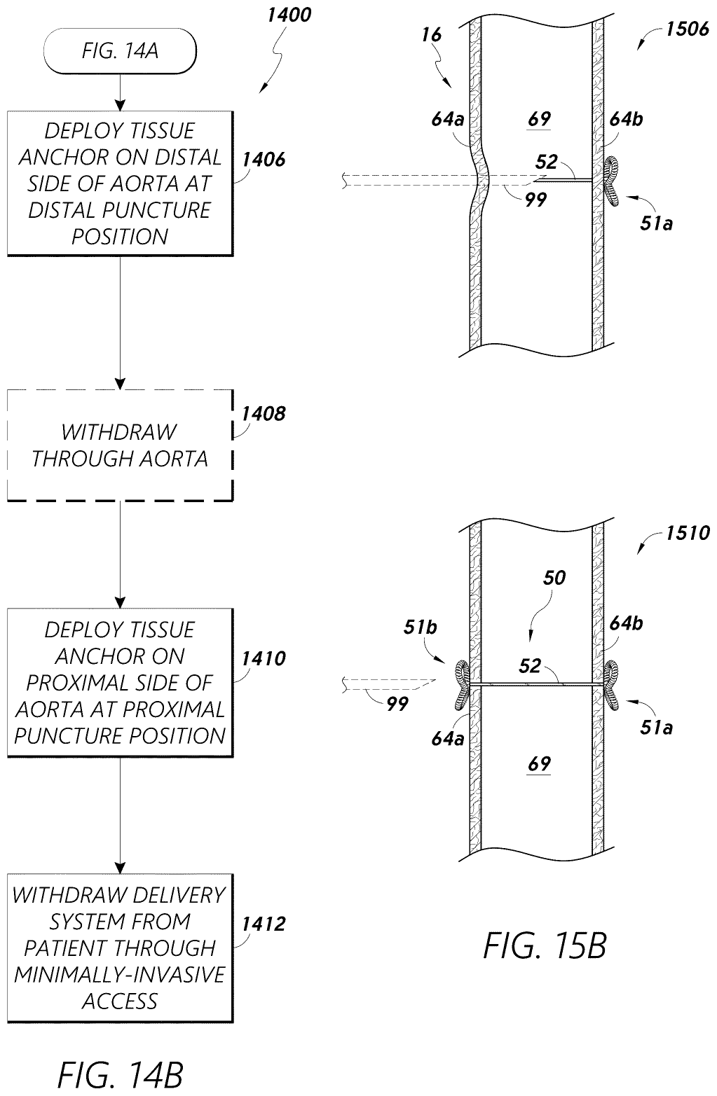

FIGS. 14A and 14B illustrate a flow diagram for a process 1400 for implanting a compliance-enhancing band implant device 50 using a minimally-invasive access in accordance with one or more examples. FIGS. 15A and 15B provide images of the compliance-enhancing implant device and certain anatomy corresponding to operations of the process 1400 of FIGS. 14A and 14B according to one or more examples. A minimally-invasive procedure for compliance-enhancing implant devices in accordance with aspects of the present disclosure may be suitable or desirable as such procedures may be implemented in a manner as to avoid breaking the blood barrier of the aorta or other target blood vessel except in the area of implantation of the device.

The process 1400 may be implemented to implant one or more compliance enhancements band implant devices in any blood vessel, such as in any part of the aorta (e.g., ascending aorta, aortic arch, descending thoracic aorta, descending abdominal aorta, etc.). At block 1402, the process 1400 involves accessing the aorta (or other target blood vessel) 16 using a minimally-invasive access opening 1501, such as through the back or flank of the patient. In some implementations, the access site 1501 may be in the fourth, fifth, or sixth intercostal space between ribs of the patient, as shown in image 1502 of FIG. 15A. A small incision may be made in the patient's back or side to provide access to the chest cavity, wherein the delivery device/system is advanced through the incision. In some implementations, an introducer or other device may be utilized for access through the incision and/or for dilating the access opening.

At block 1404, the process 1400 involves puncturing through the aorta 16 on one side 64a thereof, such as on posterior aspect of the aorta with respect to access through the back of the patient, wherein the puncture continues through the internal lumen 69 of the aorta 16 and punctures out of the opposite side 64a of the vessel, such that punctures are formed on opposite sides of the wall of the blood vessel. Such puncture may provide access to the interior 69 of the aorta, and may be made using a needle or other sharp instrument 99. It may be desirable to implement a low-profile puncture to avoid and/or reduce the risk of leakage or other injury/damage. The image 1504 shows a target aortic blood vessel 16 being punctured on one side 64a to provide access to the internal lumen 69 thereof, with the puncture instrument 99 (e.g., needle) continuing through the internal lumen 69 and out the opposite side 64b of the blood vessel.

At block 1406, the process 1400 involves deploying distal tissue anchor 51a of the band implant device on the distal side 64b of the aorta 16 in the area of the distal puncture. Image 1506 shows the deployed distal tissue anchor 51a against the outer diameter/surface 64b of the blood vessel 16. At block 1408, the process 1400 may involve withdrawing the delivery device/system back through the distal puncture and through the aorta and proximal puncture, wherein a band portion 52 of the implant is deployed from the delivery device/system 99 and deposited/left in the internal lumen 69 of the aorta 16 between the distal and proximal puncture locations.

At block 1410, the process 1400 involves deploying a proximal tissue anchor 51b of the device on a proximal side 64a of the aorta 16 against the proximal puncture opening, the band portion 52 and/or portion of the proximal anchor 51b passing through the proximal puncture. At block 1412, the process 1400 involves withdrawing the delivery system 99 from the patient through the minimally-invasive access 1501, thereby leaving/maintaining the implant device 50 in place in position through the aorta 16.

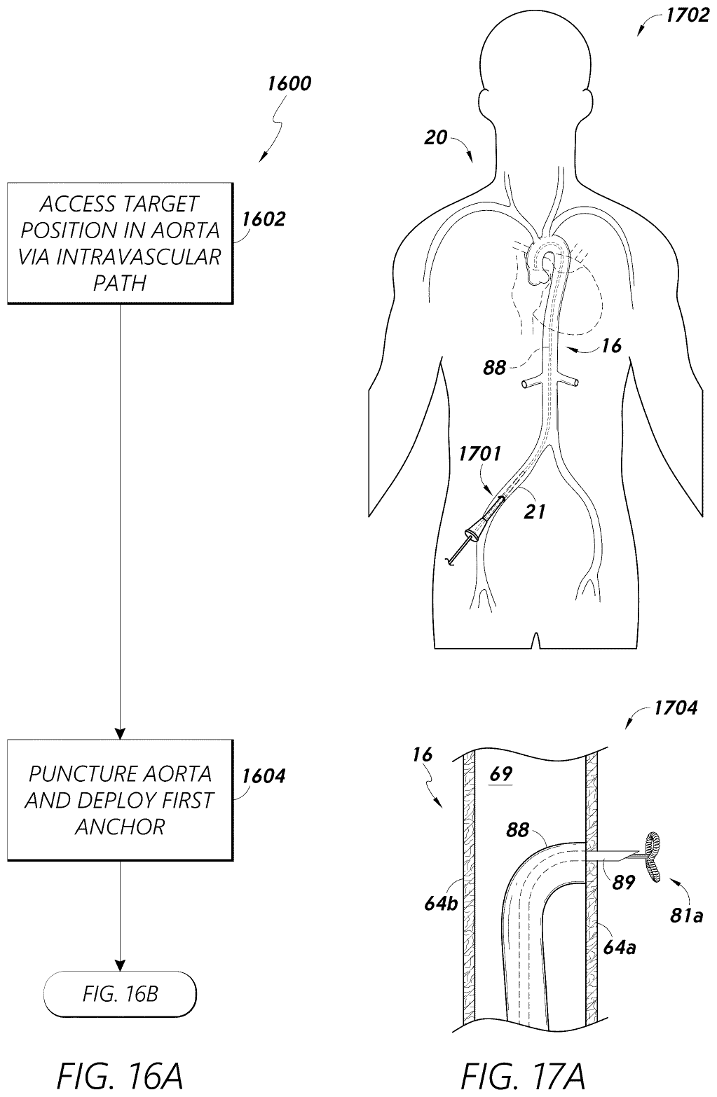

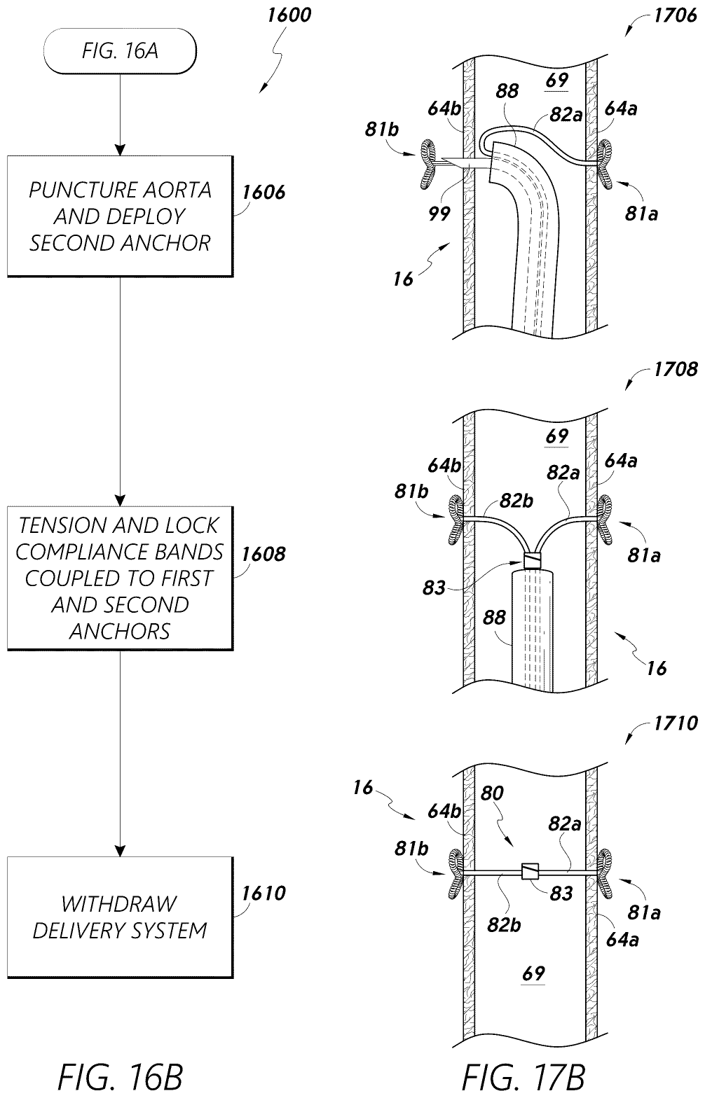

FIGS. 16A and 16B illustrate a flow diagram for a process 1600 for implanting a compliance-enhancing band implant device 80 using a transcatheter access in accordance with one or more examples. FIGS. 17A and 17B provide images of the compliance-enhancing implant device and certain anatomy corresponding to operations of the process 1600 of FIGS. 16A and 16B according to one or more examples.

At block 1602, the process 1600 involves accessing a target position in the aorta 16 of the patient 20 via a transit vascular path. Any suitable intravascular/transvascular path may be implemented that provides access to the aorta 16. For example, in some implementations, an incision 1701 may be made in the groin area to provide access to the femoral artery 21 for passage of a catheter and/or other delivery device/system 89 into the arterial system, as shown in image 1702 of FIG. 17A. In some implementations, a transcaval access path is implemented to access the target anatomy, wherein accesses is initially made via a venous blood vessel, wherein the delivery system is passed over from the venous system to the arterial system via inter vascular passage in an area where the inferior vena cava and abdominal aorta are in relatively close proximity.

At block 1604, the process 1600 involves puncturing (e.g., using a needle 99) the aorta 16 at the target site and deploying a distal/first tissue anchor 81a through the puncture on an exterior/outer surface or diameter 64a of the aorta 16 from within the blood vessel. At block 1606, the process 1600 involves puncturing the aorta on a portion 64b of the blood vessel wall that is circumferentially offset and/or opposite of the first puncture position, and deploying a second tissue anchor 81b on the outside/exterior 64b of the aorta through the second puncture opening. When implanting the second anchor 81b, the band portion(s) 82 of the implant device may be already coupled to the first 81a and second 81b tissue anchors. Alternatively, in some implementations, the tissue anchors 81 may be deployed first, after which the band portion 82 may be coupled to the tissue anchors 81. Although only two tissue anchors 81 are shown as deployed in FIG. 17B, it should be understood that any number of tissue anchors and bands may be deployed/implanted in connection with the process 1600.

In some implementations, the process 1600 may involve coupling separate band segments 82a, 82b associated with respective ones of the tissue anchors 81 to form the assembled band implant device 80. For example, as described at block 1608, the process 1600 may involve tensioning and/or locking the compliance band segments 82 coupled to the respective ones of the first 81a and second 81b tissue anchors. Such tensioning and/or locking may involve tying a knot and/or otherwise clipping, clamping, clasping, or otherwise securing/fixing the band segments 82 to one another using any suitable or desirable element or mechanism (e.g., lock 83) described herein. At block 1610, the process 1600 involves withdrawing the delivery system 88, thereby maintaining/retaining the implant 80 in place in the aorta 16 for aiding compliance and/or blood flow therein.

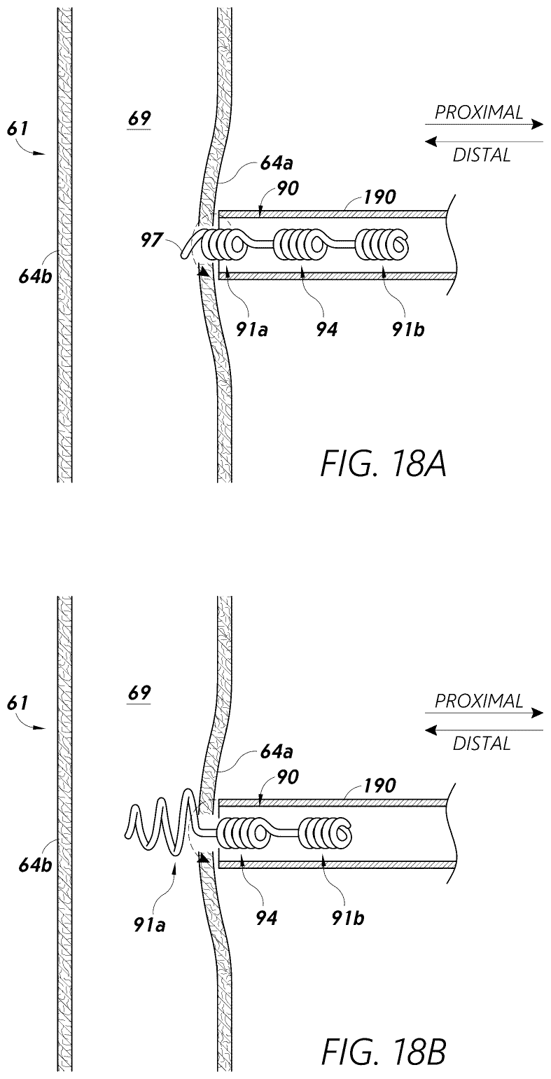

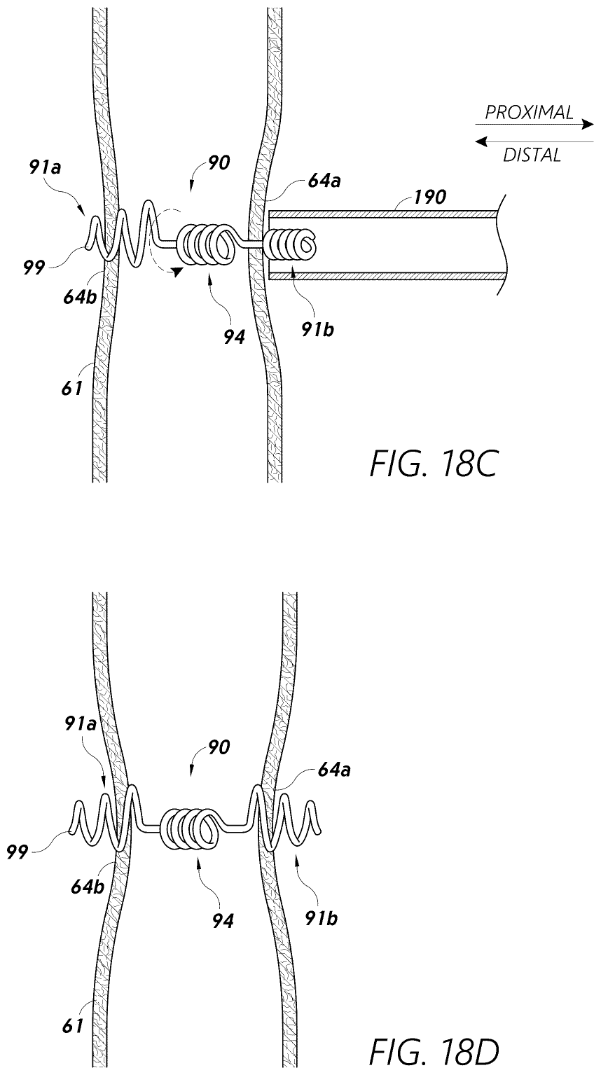

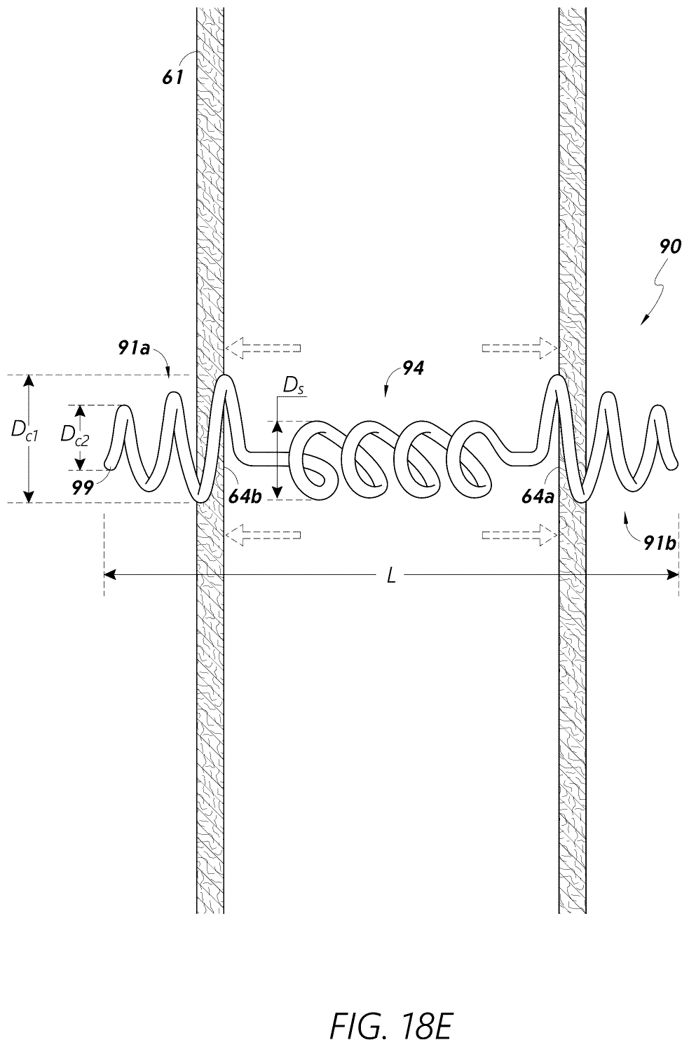

In some implementations, compliance-enhancing band implant devices in accordance with aspects of the present disclosure may comprise a single integrated coil device, wherein the coils thereof are configured to provide both anchoring and elastic stretching/contracting functionality to improve compliance. An example of such a device is shown in FIGS. 18A-18E, which show stages of a process for implanting a band implant device 90 comprising coil-type tissue anchor and elastic elements/features. The implant device 90 may be utilized or incorporated in any of the other example devices and/or processes disclosed herein. The device 90 may be formed of a wire, which may comprise any material configured to have a shape memory, such as metal (e.g., nitinol), plastic/polymer, or the like. The anchor 91 and spring 94 components can advantageously be formed of a single unitary wire form, as shown.

FIG. 18A shows the coil band implant 90 in a delivery configuration disposed within a delivery catheter/system 190, wherein the delivery device 190 is approximated to the exterior 64a of a target blood vessel 61 (e.g., aorta), such as through a minimally-invasive access as described herein. In the delivery configuration shown in FIG. 18A, the band implant 90 may be relatively elongated, wherein anchor 91 and/or compliance-enhancing 94 coils of the device 90 are compressed to some degree to reduce the profile of the device 90 during delivery; the device 90 may comprise shape-memory characteristics configured to bias the device to the expanded coiled tissue anchor and elastic spring forms of the expanded/deployed configuration thereof (see FIGS. 18D and 18E) when deployed from the delivery device/system 140. FIG. 18A shows the distal end 99 of the implant coil 90 being deployed from the distal end of the delivery device 190 and advanced in a winding manner as to cause the corkscrew form of the distal anchor portion 91a of the implant 90 to be punctured through the wall 64a of the blood vessel 61 and advanced into the lumen 69 of the blood vessel.

FIG. 18B shows the implant device 90 after it has been further wound and advanced farther into the blood vessel 61. The distal portion of the coil device 90 may form the tissue anchor 91a, and may be further advanced until it is punctured and wound through the distal wall 64b, as shown in FIG. 18C, such that the coils of the distal anchor portion 91a form/expand and spread-out on the distal/exterior side of the tissue wall 64b a manner as to provide a retention surface/form to prevent or interfere with the coil being pulled proximally back through the opening into the lumen 69 after expansion of the tissue anchor 91a on the distal side of the tissue wall 64b.

After the distal tissue anchor 91a has been advanced and disposed on the distal side of the tissue wall 64b, as shown in FIG. 18D, the delivery system 190 may be withdrawn, wherein the proximal tissue anchor 91b may further be deployed and positioned in the desired position on the proximal side of the tissue wall 64a and advanced into a desired anchoring position through the further winding of the coil and anchoring of the distal anchor 91a. The spring portion 94 of the coil 90 may have shape-memory configured to bias the spring 94 to a shortened configuration as shown in FIG. 18D, wherein increased fluid pressure in the lumen of the blood vessel 61 may cause the coil implanted to elongate in the lengthwise/axial dimension L to allow the blood vessel 61 to assume a more circular/cylindrical form having increased volume therein (see FIG. 18E), after which contraction of the coiled band portion 94 reduces the volume/area of the blood vessel and pushes blood through the area of the implant to improve compliance of the blood vessel as described in detail herein.

The tissue anchor coils 91 may be tapered in form, such that a diameter thereof is greater in a proximal area D_{c1} than at a distal end D_{c2}. The spring portion 94 may have a diameter Dₛ that is greater than or less than either the end diameter D_{c2} or the proximal diameter D_{c1} of the tissue anchor coils 91. In some examples, the spring 94 has the same diameter as at least a portion of the tissue anchor portion(s) 91, such as the proximal portion diameter D_{c1}.

### Sterilization

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

Example 1: A compliance-restoration device comprising a first elastic band biased to a contracted state and configured to longitudinally stretch and store energy in response to pulling forces, a first tissue anchor coupled to a first end of the first elastic band, and a second tissue anchor coupled to a second end of the elastic band.

Example 2: The compliance-restoration device of any example herein, in particular example 1, wherein the elastic band comprises a spring that is biased towards a longitudinally-contracted state.

Example 3: The compliance-restoration device of any example herein, in particular example 1 or 2, further comprising a hemostasis element positioned proximal of the first tissue anchor.

Example 4: The compliance-restoration device of any example herein, in particular example 3, wherein the hemostasis element comprises a pledget.

Example 5: The compliance-restoration device of any example herein, in particular example 3 or 4, wherein the hemostasis element comprises first and second patches configured to sandwich a tissue wall therebetween.

Example 6: The compliance-restoration device of any example herein, in particular examples 1-5, wherein the elastic band comprises an elastic polymer strip.

Example 7: The compliance-restoration device of any example herein, in particular examples 1-6, further comprising a second elastic band, and a third tissue anchor coupled to a first end of the second elastic band.

Example 8: The compliance-restoration device of any example herein, in particular example 7, wherein the second elastic band is coupled at a second end thereof to at least one of the first elastic band, the first tissue anchor, or the second tissue anchor.

Example 9: The compliance-restoration device of any example herein, in particular example 7 or 8, wherein the second elastic band is coupled at a second end thereof to the first tissue anchor.

Example 10: The compliance-restoration device of any example herein, in particular example 9, further comprising a third elastic band coupled at a first end thereof to the second tissue anchor.

Example 11: The compliance-restoration device of any example herein, in particular example 10, wherein the third elastic band is coupled at a second end thereof to the third tissue anchor.

Example 12: The compliance-restoration device of any example herein, in particular example 10 or 11, wherein the third elastic band is coupled at a second end thereof to a fourth tissue anchor.

Example 13: The compliance restoration device of any example herein, in particular example 12, further comprising a fourth elastic band coupled at a first end to the fourth tissue anchor and at a second end to the third tissue anchor.

Example 14: The compliance-restoration device of any example herein, in particular examples 1-13, wherein the first elastic band comprises a hook feature associated with the first end of the first elastic band, the hook feature being configured to hook onto an eyelet fastener associated with the first tissue anchor.

Example 15: The compliance-restoration device of any example herein, in particular examples 1-14, wherein the first elastic band comprises a first band segment, and a second band segment coupled to the second band segment.

Example 16: The compliance-restoration device of any example herein, in particular example 15, further comprising a lock configured to couple the first band segment to the second band segment.

Example 17: The compliance-restoration device of any example herein, in particular example 15 or 16, wherein the first end of the first elastic band is associated with the first band segment, and the second end of the first elastic band is associated with the second band segment.

Example 18: The compliance-restoration device of any example herein, in particular examples 1-17, wherein the first tissue anchor comprises a suture knot.

Example 19: The compliance-restoration device of any example herein, in particular examples 1-8, wherein the first tissue anchor comprises a ring having one or more spokes.

Example 20: The compliance-restoration device of any example herein, in particular example 19, wherein the one or more spokes are formed of suture.

Example 21: The compliance-restoration device of any example herein, in particular examples 1-20, wherein the first tissue anchor comprises a patch.

Example 22: The compliance-restoration device of any example herein, in particular examples 1-21, wherein the first tissue anchor comprises a wire formed into a ring.

Example 23: The compliance-restoration device of any example herein, in particular example 22, wherein the first tissue anchor comprises suture spokes coupled to the ring at circumferentially-offset positions.

Example 24: A compliance-enhancing band device comprising a first coil tissue anchor, a second coil tissue anchor, and a spring connected between first coil tissue anchor and the second coil tissue anchor.

Example 25: The band device of any example herein, in particular example 24, wherein the first coil tissue anchor, the second coil tissue anchor, and the spring are formed of a unitary wire form.

Example 26: The band device of any example herein, in particular example 24 or 25, wherein the first coil tissue anchor, the second coil tissue anchor, and the spring are formed from a single wire.

Example 27: The band device of any example herein, in particular examples 24-26, wherein the first coil tissue anchor comprises a tapered coil.

Example 28: The band device of any example herein, in particular example 27, wherein the tapered coil is tapered towards an end of the band device.

Example 29: The band device of any example herein, in particular example 27 or 28, wherein the first coil tissue anchor has a maximum diameter that is greater than a maximum diameter of the spring.

Example 30: A method of adding compliance to a blood vessel, the method comprising accessing a target segment of a blood vessel through an intravascular access path, anchoring a first tissue anchor to a first tissue wall portion of the target segment of the blood vessel, anchoring a second tissue anchor to a second tissue wall portion of the target segment of the blood vessel, and coupling a first elastic band between the first tissue anchor and the second tissue anchor, the first elastic band being configured to be pulled to an elongated configuration by the first and second tissue anchors in response to fluid pressure within the blood vessel.

Example 31: The method of any example herein, in particular example 30, wherein said coupling the first elastic band between the first tissue anchor and the second tissue anchor is performed prior to said accessing the target segment of the blood vessel.

Example 32: The method of any example herein, in particular example 30 or 31, wherein said coupling the first elastic band between the first tissue anchor and the second tissue anchor is performed after said anchoring the first tissue anchor and said anchoring the second tissue anchor.

Example 33: The method of any example herein, in particular examples 30-32, wherein said coupling the first elastic band between the first tissue anchor and the second tissue anchor comprises coupling a first band segment that is coupled to the first tissue anchor to a second band segment that is coupled to the second tissue anchor using a locking means.

Example 34: The method of any example herein, in particular example 33, wherein the locking means comprises at least on of a clip, a clamp, a knot, a hook, or a loop.

Example 35: The method of any example herein, in particular examples 30-34, wherein the first elastic band comprises a spring that is biased towards a longitudinally-contracted state.

Example 36: The method of any example herein, in particular examples 30-35, wherein the first elastic band comprises an elastic polymer strip.

Example 37: The method of any example herein, in particular examples 30-36, further comprising placing a hemostasis patch against an inner wall of the first tissue wall portion.

Example 38: The method of any example herein, in particular examples 30-37, further comprising anchoring a third tissue anchor to a third tissue wall portion of the target segment of the blood vessel, and coupling a second elastic band to the third tissue anchor.

Example 39: The method of any example herein, in particular example 38, further comprising coupling the second elastic band to at least one of the first elastic band, the first tissue anchor, or the second tissue anchor.

Example 40: The method of any example herein, in particular example 38 or 39, further comprising coupling a third elastic band to the third tissue anchor.

Example 41: The method of any example herein, in particular example 40, further comprising coupling the third elastic band to the second tissue anchor.

Example 42: The method of any example herein, in particular example 40, wherein the first elastic band, the second elastic band, and the third elastic band are each segments of a single integrated band.

Example 43: The method of any example herein, in particular example 42, wherein the single integrated band can slide longitudinally through band-engagement features of the first tissue anchor, the second tissue anchor, and the third tissue anchor.

Example 44: The method of any example herein, in particular examples 40-43, further comprising anchoring a fourth tissue anchor to a fourth tissue wall portion of the target segment of the blood vessel, and coupling a fourth elastic band to the fourth tissue anchor.

Example 45: The method of any example herein, in particular example 44, further comprising coupling the third elastic band to the fourth tissue anchor, and coupling the fourth elastic band to the second tissue anchor.

Example 46: The method of any example herein, in particular example 45, wherein the first elastic band, the second elastic band, the third elastic band, and the fourth elastic band are each segments of a single integrated band.

Example 47: The method of any example herein, in particular example 46, wherein the single integrated band can slide longitudinally through band-engagement features of the first tissue anchor, the second tissue anchor, the third tissue anchor, and the fourth tissue anchor.

Example 48: A compliance-restoration device comprising a first tissue anchor, a second tissue anchor, a third tissue anchor, each of the first, second, and third tissue anchors including a distal retention member and a proximal band-engagement loop, and an elastic band disposed through the respective band-engagement loops of the first, second, and third tissue anchors.

Example 49: The compliance-restoration device of any example herein, in particular example 48, wherein the elastic band form a loop.

Example 50: The compliance-restoration device of any example herein, in particular example 48 or 49, wherein the elastic band comprises a plurality of longitudinally-offset springs, each of the plurality of longitudinally-offset springs being disposed between a pair of the first, second, and third tissue anchors.

Example 51: The compliance-restoration device of any example herein, in particular examples 48-50, further comprising a fourth tissue anchor, wherein the elastic band is disposed through a band-engagement loop of the fourth tissue anchor.

Example 52: The compliance-restoration device of any example herein, in particular example 51, wherein first and second ends of the band are coupled together to form a loop.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of adding compliance to a blood vessel, the method comprising:
accessing a target segment of a blood vessel;
forming a first puncture through a first wall segment of the blood vessel;
forming a second puncture through a second wall segment of the blood vessel, the second wall segment being axially aligned with the first wall segment at a target axial position in the blood vessel and diametrically opposite the first wall segment;
deploying a first tissue anchor on an outside of the blood vessel in an area of the first puncture;
deploying a second tissue anchor on the outside of the blood vessel in an area of the second puncture; and
tensioning a first elastic band coupled to the first tissue anchor and the second tissue anchor, thereby causing the first elastic band to reshape the target segment of the blood vessel to a non-circular cross-sectional shape to push blood through the blood vessel.

2. The method of claim 1, wherein forming the first puncture and the second puncture involves:
forming a minimally-invasive access opening through a back or flank of a patient to provide access to a chest cavity of the patient; and
passing a delivery device through the minimally-invasive access opening;
wherein the first puncture is formed using the delivery device.

3. The method of claim 2, wherein forming the first puncture and the second puncture further involves:
deploying a needle from the delivery device;
advancing the needle through the first wall segment from the outside of the blood vessel;
advancing the needle further through an internal lumen of the blood vessel; and
advancing the needle further through the second wall segment from within the blood vessel.

4. The method of claim 1, wherein tensioning the first elastic band is achieved by deploying the second tissue anchor.

5. The method of claim 1, wherein forming the first puncture and the second puncture involves:
advancing a delivery device within an internal lumen of the blood vessel via an endovascular access path;
deploying a cutting tool from the delivery device;
cutting the first puncture in the first wall segment of the blood vessel from within the internal lumen of the blood vessel; and
cutting the second puncture in the second wall segment of the blood vessel from within the internal lumen of the blood vessel.

6. The method of claim 5, wherein:
tensioning the first elastic band involves fixing a first segment of the first elastic band to a second segment of the first elastic band within the internal lumen of the blood vessel;
the first segment of the first elastic band is attached to the first tissue anchor; and
the second segment of the first elastic band is attached to the second tissue anchor.

7. The method of claim 1, wherein:
the first tissue anchor includes a first anchor portion positioned on the outside of the blood vessel and a first band-engagement loop positioned in an internal lumen of the blood vessel;
the second tissue anchor includes a second anchor portion positioned on the outside of the blood vessel and a second band-engagement loop positioned in an internal lumen of the blood vessel; and
tensioning the first elastic band involves coupling the first elastic band to at least one of the first band-engagement loop or the second band-engagement loop.

8. The method of claim 1, further comprising:
coupling a second elastic band to the first tissue anchor; and
coupling a third elastic band to the second tissue anchor;
wherein the second elastic band and the third elastic band are coupled to a third tissue anchor deployed on the outside of the blood vessel.

9. A method of adding compliance to a blood vessel, the method comprising:
accessing a target segment of a blood vessel through an intravascular access path;
anchoring a first tissue anchor to a first tissue wall portion of the target segment of the blood vessel;
anchoring a second tissue anchor to a second tissue wall portion of the target segment of the blood vessel; and
coupling a first elastic band between the first tissue anchor and the second tissue anchor, the first elastic band being configured to be pulled to an elongated configuration by the first tissue anchor and the second tissue anchor in response to fluid pressure within the blood vessel.

10. The method of claim 9, wherein coupling the first elastic band between the first tissue anchor and the second tissue anchor is performed prior to accessing the target segment of the blood vessel.

11. The method of claim 9, wherein coupling the first elastic band between the first tissue anchor and the second tissue anchor is performed after anchoring the first tissue anchor and anchoring the second tissue anchor.

12. The method of claim 9, wherein coupling the first elastic band between the first tissue anchor and the second tissue anchor comprises coupling a first band segment that is coupled to the first tissue anchor to a second band segment that is coupled to the second tissue anchor using a locking means.

13. The method of claim 12, wherein the locking means comprises at least on of a clip, a clamp, a knot, a hook, or a loop.

14. The method of claim 9, wherein the first elastic band comprises a spring that is biased towards a longitudinally-contracted state.

15. The method of claim 9, wherein the first elastic band comprises an elastic polymer strip.

16. The method of claim 9, further comprising placing a hemostasis patch against an inner wall of the first tissue wall portion.

17. The method of claim 9, further comprising:
anchoring a third tissue anchor to a third tissue wall portion of the target segment of the blood vessel;
coupling a second elastic band to the third tissue anchor;
coupling the second elastic band to the first tissue anchor;
coupling a third elastic band to the third tissue anchor; and
coupling the third elastic band to the second tissue anchor.

18. The method of claim 17, wherein:
the first elastic band, the second elastic band, and the third elastic band are each segments of a single integrated band; and
the single integrated band can slide longitudinally through band-engagement features of the first tissue anchor, the second tissue anchor, and the third tissue anchor.

19. A method of adding compliance to a blood vessel, the method comprising:
cutting a minimally-invasive access opening through a back or flank of a patient;
accessing a target segment of an aorta of the patient through the minimally-invasive access opening and between ribs of the patient;
puncturing the aorta using a cutting tool from outside the aorta at a first puncture site;
advancing the cutting tool through an internal lumen of the aorta;
puncturing the aorta using the cutting tool from inside the aorta at a second puncture site axially aligned with the first puncture site with respect to an axis of the aorta;
deploying a first tissue anchor on an outside surface of the aorta at the second puncture site using a delivery device;
withdrawing the delivery device through the internal lumen of the aorta, thereby deploying an elastic band attached to the first tissue anchor in the internal lumen of the aorta;
deploying a second tissue anchor on an outside surface of the aorta at the first puncture site using the delivery device; and
withdrawing the delivery device from the patient through the minimally-invasive access opening.

20. The method of claim 19, further comprising reshaping the aorta to a non-circular cross-sectional shape using the elastic band.
